(19) United States
(12) Patent Application Publication
(10) Pub. No.: US 2018/0147161 A1
(43) Pub. Date: May 31, 2018

(54) MULTIDRUG BRITTLE MATRIX COMPOSITIONS

(71) Applicant: BOARD OF REGENTS, THE UNIVERSITY OF TEXAS SYSTEM, Austin, TX (US)

(72) Inventors: Robert O. WILLIAMS, III, Austin, TX (US); Alan WATTS, Austin, TX (US); Jay PETERS, San Antonio, TX (US)

(73) Assignee: BOARD OF REGENTS, THE UNIVERSITY OF TEXAS SYSTEM, Austin, TX (US)

(21) Appl. No.: 15/570,828

(22) PCT Filed: Sep. 2, 2015
(86) PCT No.: PCT/US2015/048093

§ 371 Date: Oct. 31, 2017

Related U.S. Application Data
(60) Provisional application No. 62/156,052, filed on May 1, 2015.

(51) Int. Cl. A61K 31/137; A61K 31/167; A61K 31/58; A61K 31/439; A61P 11/00; A61K 9/14; A61K 9/00

(52) U.S. Cl. CPC A61K 31/137; A61K 31/167; A61K 31/58; A61K 2300/00; A61P 11/00; A61K 9/145; A61K 9/0075; A61K 31/439

(57) ABSTRACT

Dual and triple therapy combinations of drugs formulated as brittle matrix particles with a high surface area are provided herein. These particle formulations may be used in inhalation or aerosol administration techniques to provide the drug combinations to the lungs. In some aspects, these compositions may be used to treat a respiratory disease or disorder such as asthma or COPD.

This application claims the benefit of priority to U.S. Provisional Application Ser. No. 62/156,052 filed on May 1, 2015, the entire contents of which are hereby incorporated by reference.

### BACKGROUND OF THE INVENTION

### 1. Field of the Invention

The present invention relates generally to the field of pharmaceutical compositions. More particularly, it concerns pharmaceutical compositions with two or three active pharmaceutical ingredients prepared as brittle matrix particles.

### 2. Description of Related Art

Asthma is a serious health problem and presents a significant burden to families throughout the world. It places severe limits on daily life and can be fatal. People of all ages are affected by this chronic airway disorder, and the incidence of asthma is increasing in most countries especially among children (Bateman et al., 2008). One of the indicated treatment methods for asthma is corticosteroids (ICS) with or without long acting β2-agonists (LABA) (Silvasti et al., 1996; Chowdhury and Pan, 2010). Additionally, the ICS/LABA combination is an effective therapy for chronic obstructive pulmonary disease (COPD) that, compared to other therapies, has been shown to reduce exacerbations, hospitalizations, emergency room visits and health care costs (Mapel et al., 2010).

The ability of long-acting β2-agonists to prime glucocorticosteroid receptors improves the activity of inhaled corticosteroids. It is important that inhaled combinations are co-deposited within the lungs, since this synergy occurs at the cellular/molecular level (Greening et al., 1994). In addition, salmeterol xinafoate (SX) is sparingly soluble and mometasone furoate (MF) is practically insoluble in water (Jouyban-Gharamaleki et al., 2001; Zitt et al., 2007), and their low solubility limits absorption and bioavailability, which impacts their clinical use. Different crystal engineering strategies have been effective at modifying the physicochemical properties and enhancing oral bioavailability for specific compounds, more recently especially co-crystal (Sowa et al., 2014; Évora et al., 2011) and co-amorphous systems (Lobmann et al., 2013; Shayanfar et al., 2013), which could achieve co-deposition for formulations consisted of more than one API component.

However, presently there is no fixed dose combination product marketed for inhalation containing SX and MF contained within the same particle. Having SX and MF within the same particle allows deposition of the APIs at the same target site within the lungs. Presently available products containing two APIs are typically micronized separately and remain as discrete particles in the formulation and device (Patil et al., 2012). Furthermore, micronized API particles can possess different crystallography and morphology, resulting in differences in aerodynamic performance (Parikh et al., 2012). Researchers have presented a solution to the problem of traditional blends by engineering multiple drugs into a single particle (Parikh et al., 2012; Weers and Tarara, 2014). Thus, new compositions which contain multiple active pharmaceutical ingredients with the same particle are needed.

### SUMMARY OF THE INVENTION

The present disclosure provides pharmaceutical compositions formulated as a brittle matrix particle for intranasal, via inhalation, via aerosol, or administered to the lungs. In some embodiments, these compositions comprise a long acting β-agonist and a corticosteroid or a long acting β-agonist, a long acting muscarinic antagonist, and a corticosteroid. These compositions may be used to treat a respiratory disease or disorder including but not limited to asthma or chronic obstructive pulmonary disease (COPD).

In some aspects, the present disclosure provides pharmaceutical compositions comprising either:
a. a triple therapy comprising a therapeutically effective amount of an active pharmaceutical ingredient from each of the following groups:
   i. a long acting β-agonist (LABA);
   ii. a long acting muscarinic antagonist (LAMA); and
   iii. a cortiscosteroid (CS); or
b. a dual therapy comprising a therapeutically effective amount of an active pharmaceutical ingredient from both of the following groups:
   i. a long acting β-agonist (LABA), and
   ii. a corticosteroid (CS);
      wherein the pharmaceutical composition is formulated as a brittle matrix particle having a specific surface area of greater than 5 m²/g. In some embodiments, the pharmaceutical compositions further comprise one or more excipients. The excipients that may be used include a sugar, sugar derivative, or an amino acid. The excipients may be a sugar or a sugar derivative such as a lactose, mannitol, or trehalose. In other embodiments, the excipients are an amino acid such as glycine.

In some embodiments, the long acting β-agonists are a salmeterol or formoterol salt such as salmeterol xinafoate or formoterol fumarate. In some embodiments, the corticosteroids are mometasene furoate or budesomide. In some embodiments, the long acting muscarinic antagonists are a tiotropium salt such as tiotropium bromide.

The dual therapies may comprise a weight ratio of the long acting β-agonist to the corticosteroid from about 1:0.1 to about 1:100 in the composition. In some embodiments, the dual therapies have a weight ratio of about 5:22 of the long acting β-agonist to the corticosteroid. In other embodiments, the triple therapies comprise a weight ratio of the long acting β-agonist to the long acting muscarinic antagonist to the corticosteroid from about 1:0.1:0.1 to about 1:100:100 in the composition such as about 1:2:35.5 of the long acting β-agonist to the long acting muscarinic antagonist to the corticosteroid. In some embodiments, the triple therapies comprise a weight ratio of the long acting β-agonist to the long acting muscarinic antagonist from about 1:0.1 to about 1:100 in the composition such as about 1:2 of the long acting β-agonist to the long acting muscarinic antagonist. In some embodiments, the triple therapies comprise a weight ratio of the long acting β-agonist to the corticosteroid from about 1:0.1 to about 1:100 in the composition such as about 2:70 of the long acting β-agonist to the corticosteroid. In some embodiments, the triple therapies comprise a weight ratio of the long acting muscarinic antagonist to the corticosteroid from about 1:0.1 to about 1:1000 in the composition such as about 4:70 of the long acting muscarinic antagonist to the corticosteroid. The pharmaceutical compositions may have a molar ratio of the dual therapy or the triple therapy to the excipient of from about 1:0 to about 1:9 in the composition such as about 1:1 of the dual therapy or the triple therapy to the excipient.

In some aspects, the pharmaceutical compositions are formulated as a unit dose. The unit dose of the pharmaceutical compositions may comprises a dose of the long acting β-agonist from about 1 to about 500 μg such as about 50 μg when the long acting β-agonist is salmeterol xinafoate or about 4.5 μg when the long acting β-agonist is formoterol fumarate. In some embodiments, the unit dose of the pharmaceutical compositions comprises a dose of the long acting muscarinic antagonist from about 1 to about 100 μg. such as a unit dose of about 9 μg. In some embodiments, the unit dose of the pharmaceutical compositions comprises a dose of the corticosteroid from about 1 to about 1000 μg such as about 220 μg when the corticosteroid is mometasene furoate or about 160 μg when the corticosteroid is budesomide.

In some aspects, the pharmaceutical compositions re formulated for administration: intranasally, via aerosol, to the lungs, or via inhalation. The pharmaceutical compositions may be formulated for use in an inhaler including a metered dose inhaler, a dry powder inhaler, a single dose inhaler, a multi-unit dose inhaler, a nebulizer, or a pressurized metered dose inhaler.

In some aspects, the pharmaceutical compositions are the dual therapy comprising salmeterol xinafoate and mometasene furoate. In other aspects, the pharmaceutical compositions are the triple therapy comprising formoterol fumarate, tiotropium bromide, and budesomide.

In some embodiments, the pharmaceutical compositions have a specific surface area from about 5 m²/g to about 1000 m²/g, from about 10 m²/g to about 500 m²/g, or from about 20 m²/g to about 250 m²/g. In some embodiments, the pharmaceutical compositions have a total emitted dose (TED) of greater than 85% such as from about 90% to about 100%.

In some embodiments, the pharmaceutical composition is free of any impurities. The pharmaceutical composition may be substantially free of polyvinylpyrrolidone, polyvinylalcohol, polyacrylate, or polystyrene. The pharmaceutical composition may be essentially free of polyvinylpyrrolidone, polyvinylalcohol, polyacrylate, or polystyrene. In some embodiments, the pharmaceutical composition is essentially free of any polymeric excipients. The pharmaceutical composition may be substantially free of poloxamers, polyethylene glycol, or polypropylene glycol. The pharmaceutical composition may be essentially free of poloxamers, polyethylene glycol, or polypropylene glycol. In some embodiments, the pharmaceutical composition is essentially free of any surfactants. In some embodiments, the pharmaceutical composition is free of other compounds beyond the excipient and the active pharmaceutical composition.

In still another aspect, the present disclosure provides methods of treating or preventing a respiratory disease or disorder in a patient in need thereof comprising administering to the patient a therapeutically effective amount of a pharmaceutical composition disclosed herein. The respiratory diseases or disorders that may be treated include a disorder involving inflammation of the lungs or sinuses. Additionally, the respiratory disease or disorder may be asthma or chronic obstructive pulmonary disease. In some embodiments, the pharmaceutical composition is administered via inhalation. The therapeutically effective amount may be administered to the patient in one inhalation. or in 2 or more inhalations. In some embodiments, the therapeutically effective amount is administered in 2, 3, or 4 inhalations. In some embodiments, the method comprises administering the therapeutically effective amount to the patient once a day. In other embodiments, the method comprises administering the therapeutically effective amount to the patient two or more times a day.

In still yet another aspect, the present disclosure provides methods of preparing a brittle matrix pharmaceutical composition comprising:
(A) admixing two or more active pharmaceutical agents into a solvent wherein the solvent comprises an organic solvent and water to form a pharmaceutical composition wherein the pharmaceutical composition comprises an amount of the active pharmaceutical agents in the solvent from about 0.01% to about 10% (w/v);
(B) applying the pharmaceutical composition to a rotating surface wherein the surface is at a temperature from about −70° C. to about −120° C.; and
(C) freezing the pharmaceutical composition to form a brittle matrix pharmaceutical composition.

In some embodiments, the active pharmaceutical ingredients are selected from a long acting β-agonist, a long acting muscarinic antagonist, and a corticosteroid. The methods may also further comprise adding one or more excipients to the pharmaceutical composition. In some embodiments, the method further comprises lyophilizing the brittle matrix pharmaceutical composition. In some embodiments, the amount is from about 0.01% (w/v) to about 6% (w/v). In some embodiments, the amount is from about 0.1% (w/v) to about 5% (w/v).

In still yet another aspect, the present disclosure provides brittle matrix pharmaceutical compositions prepared by the method comprising:
(A) admixing two or more active pharmaceutical agents into a solvent wherein the solvent comprises an organic solvent and water to form a pharmaceutical composition wherein the pharmaceutical composition comprises an amount of the active pharmaceutical agents in the solvent from about 0.01% to about 10% (w/v);
(B) applying the pharmaceutical composition to a rotating surface wherein the surface is at a temperature from about −70° C. to about −120° C.; and
(C) freezing the pharmaceutical composition to form a brittle matrix pharmaceutical composition.

In some embodiments, the active pharmaceutical ingredients are selected from a long acting β-agonist, a long acting muscarinic antagonist, and a corticosteroid. The method may also further comprises adding one or more excipients to the pharmaceutical composition. In some embodiments, the method further comprises lyophilizing the brittle matrix pharmaceutical composition. In some embodiments, the amount is from about 0.01% (w/v) to about 6% (w/v). In some embodiments, the amount is from about 0.1% (w/v) to about 5% (w/v).

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to effect such treatment for the disease.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

The term “free of” is used to imply a particle which contains at least 95% of the listed components and less than 5% of the components to which composition or particle is free of. The term “substantially free of” is used to 98% of the listed components and less than 2% of the components to which composition or particle is substantially free of. In some embodiments, the term “essentially free of” is used to describe a particle or composition which contains at least 99% of the listed components and contains less than 1% of any components of the components to which it is essentially free of.

As used herein the terms dual therapy or triple therapy comprise at least the two or three active pharmaceutical ingredients listed therein. It is also contemplated that these terms are not meant to imply that these therapies comprise only two or three active pharmaceutical ingredients but rather that they comprise at least two or at least three active pharmaceutical ingredients.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows geometric particle size distribution of BMP before and after dosing from a HandiHaler®.

FIG. 2 shows aerodynamic particle size distribution of a triple combination aerosol using micronized (left) and BMP (right) formulations with the values of formoterol, tiotropium, and budesonide shown from left to right for each site. Aerosols were generated using a HandiHaler® at 51 l/min.

FIG. 3 shows modulated DSC profiles (from bottom to top) BMP SXMFTre, BMP SXMFGly, BMP SXMFMan, BMP SXMFLac and BMP SXMF. T_{g} was shown in small window.

FIGS. 4A & 4B show powder X-ray diffraction patterns for the compositions. FIG. 4A shows the X-ray powder diffraction patterns for (from bottom to top): micronized SXMF, BMP SXMF, micronized SXMFLac, BMP SXMFLac, micronized SXMFMan, BMP SXMFMan, micronized SXMFGly, BMP SXMFGly, micronized SXMFTre, TFF SXMFTre. FIG. 4B shows the X-ray powder diffraction patterns of BMP combinations stored at 25° C./30% RH for 6 months (from bottom to top): BMP SXMF, BMP SXMFLac, BMP SXMFMan, BMP SXMFGly and BMP SXMFTre.

FIGS. 5A & 5B show the SEM images of (FIG. 5A) Micronized SXMF (FIG. 5B) BMP SXMF.

FIGS. 6A & 6B show the Fourier transform infrared spectroscopy of (FIG. 6A) micronized single ingredient (FIG. 6B) TFF processed co-drug deposition amorphous forms.

FIG. 7 shows the isotherms of sorption (-) and desorption (---) of (□) BMP SXMF, (⋄) BMP SXMFLac, (Δ) BMP SXMFMan, (×) BMP SXMFGly and (∘) BMP SXMFTre produced after one cycle between 0% and 90%.

FIGS. 8A-8E show the aerodynamic diameter distribution of SX and MF for (FIG. 8A) micronized SXMF and BMP SXMF, (FIG. 8B) micronized SXMFLac and BMP SXMFLac, (FIG. 8C) micronized SXMFMan and BMP SXMFMan, (FIG. 8D) micronized SXMFGly and BMP SXMFGly, and (FIG. 8E) micronized SXMFTre and BMP SXMFTre after produced by Miat at 90 L/min. Values were expressed as mean±SD (n=3).

FIG. 9 shows the aerodynamic diameter distribution of SX and MF for micronized SXMF and TFF SXMF after produced by Insufflator at 51 L/min.

FIGS. 10A-10E show the SEM images of (FIG. 10A) budesonide, (FIG. 10B) tiotropium bromide and (FIG. 10C) formoterol fumarate at magnification of 10.0K, (FIG. 10D) mannitol, and (FIG. 10E) lactose monohydrate (1.0K).

FIGS. 11A-11E show the SEM images of (FIG. 11A) jet milled budesonide (FIG. 11B) jet milled tiotropium, (FIG. 11C) jet milled formoterol and the physical mixtures (FIG. 11D) BTF_Lac_PM and (FIG. 11E) BTF_Man_PM (5.0K).

FIGS. 12A-12D show the SEM images of TFF formulations (FIG. 12A) Bud_Lac, (FIG. 12B) Bud_Man, (FIG. 12C) Tio_Lac, and (FIG. 12D) Tio_Man at magnification 1.0K.

FIGS. 13A-13D show the SEM images of TFF formulations (FIG. 13A) For_Lac, (FIG. 13B) For_Man, (FIG. 13C) BTF_Lac, and (FIG. 13D) BTF_Man at magnification 1.0K.

FIG. 14 shows the modulated DSC heat flow thermograms of unprocessed lactose monohydrate, mannitol, budesonide, tiotropium and formoterol, physical mixture formulation of jet milled budesonide, tiotropium and formoterol with mannitol (BTF_Man) and with lactose (BTF_Lac), and jet milled tiotropium, formoterol and budesonide.

FIG. 15 shows the modulated DSC heat flow thermograms of unprocessed lactose monohydrate, mannitol, budesonide, formoterol and tiotropium, TFF formulations of Bud_Man, For_Man, Tio_Man and BTF_Man.

FIG. 16 shows the modulated DSC heat flow thermograms of unprocessed lactose monohydrate, mannitol, budesonide, formoterol and tiotropium, TFF formulations of BTF_Lac, Bud_Lac, For_Lac, and Tio_Lac.

FIGS. 17A-17N show the powder x-ray pattern of (FIG. 17A) mannitol, (FIG. 17B) lactose monohydrate, (FIG. 17C) budesonide, (FIG. 17D) tiotropium, (FIG. 17E) formoterol, (FIG. 17F) Bud_Man, (FIG. 17G) Tio_Man, (FIG. 17H) For_Man, (FIG. 17I) BTF_Man, (FIG. 17J) Bud_Lac, (FIG. 17L) Tio_Lac, (FIG. 17M) For_Lac and (FIG. 17N) BTF_Lac.

FIGS. 18A-18H show the powder x-ray pattern of (FIG. 18A) lactose monohydrate, (FIG. 18B) mannitol, (FIG. 18C) budesonide, (FIG. 18D) jet milled budesonide, (FIG. 18E) tiotropium, (FIG. 18F) jet milled tiotropium, (FIG. 18G) formoterol, and (FIG. 18H) jet milled formoterol.

FIG. 19 shows the FTIR scans of TFF formoterol_lactose, TFF budesonide_lactose, TFF tiotropium_lactose, physical mixture of triple drug combination, TFF triple drug combination, formoterol, budesonide, tiotropium, TFF lactose and lactose.

FIG. 20 shows the FTIR scans of TFF formoterol_mannitol, TFF budesonide_mannitol, TFF tiotropium_mannitol, TFF triple drug combination, formoterol, budesonide, tiotropium, physical mixture of triple drug combination, TFF mannitol and mannitol.

FIGS. 21A & 21B show (FIG. 21A) the aerodynamic particle size distribution of TFF triple combo BTF_Lac formulations deposited on a next generation cascade impactor and (FIG. 21B) the aerodynamic particle size distribution of TFF triple combo BTF_Man formulations deposited on a next generation cascade impactor.

FIGS. 22A & 22B show (FIG. 22A) the aerodynamic particle size distribution of TFF single drug formulations For_Lac, Tio_Lac and Bud_Lac, deposited on a next generation cascade impactor and (FIG. 22B) the aerodynamic particle size distribution of TFF single drug formulations For_Man, Tio_Man and Bud_Man, deposited on a next generation cascade impactor.

FIGS. 23A & 23B show (FIG. 23A) the aerodynamic particle size distribution of triple combo physical mixture BTF_Lac_PM formulations deposited on a next generation cascade impactor and (FIG. 23B) the aerodynamic particle size distribution of triple combo physical mixture BTF_Man_PM formulations deposited on a next generation cascade impactor.

### DESCRIPTION OF ILLUSTRATIVE EMBODIMENTS

In some aspects, the present disclosure provides a pharmaceutical composition formulated as a brittle matrix particle with two or more active pharmaceutical ingredient (API) for administration via inhalation, via aerosol, or other methods of delivering the compound to the lungs. The pharmaceutical compositions described herein may comprise a high surface area and a high total emitted dose. In some embodiments, these pharmaceutical compositions have increased effectiveness in delivering two or more active pharmaceutical ingredients to the lungs of a patient.

### I. BRITTLE MATRIX PARTICLES

In some aspects, the present disclosure provides the use of brittle matrix particles (BMP). The brittle matrix particles that may be used herein are characterized by their low density configuration with a high surface area and high porosity. These brittle matrix particles may be prepared using convention methods such as spray freeze drying or thin film freezing as described herein and in U.S. Patent Application No. 2010/0221343 and Watts, et al., 2013, both of which are incorporated herein by reference. In some embodiments, thin film freezing is used to prepare the brittle matrix particles described herein. After freezing, these particles may be further subjected to drying to obtain a dry powder suitable for aerosol administration. The brittle matrix particles may be dried through lyophilization and other methods known to those of skill in the art. Without wishing to be bound by any theory, the brittle matrix particles and the fast freezing drying methods allow the mixing of the particles while maintaining the homogeneity of the mixture while preventing segregation of the different components. The improved homogeneity may also be exhibited during the aerosolization process.

In some aspects, the brittle matrix particles are prepared using thin film freezing (TFF) methods. Such preparation may be used in a manner to allow for the co-deposition of two or more active pharmaceutical ingredients (APIs) and one or more excipients to form a pharmaceutical composition. In some embodiments, the methods comprise dissolving the pharmaceutical composition in a solvent. Some solvents which may be used in the methods described herein include water, an organic solvent, or a mixture thereof. The organic solvents that may be used herein include polar organic solvents such an alcohol, a heterocyclic compound, an alkylnitrile, or a mixture thereof. Some non-limiting examples of polar organic solvents include methanol, ethanol, isopropanol, tert-butanol (tertiary butanol), dimethylsulfoxide, dimethylformamide, 1,4-dioxane, or acetonitrile. In some aspects, mixtures of these solvents are contemplated. Such mixtures may comprise one or more organic solvents with water. One non-limiting example of these mixtures includes the solvent mixture of tert-butanol, 1,4-dioxane, acetonitrile, and water. The solvent mixture may comprise a mixture of tertiary butanol, 1,4-dioxane, acetonitrile, and purified water in a ratio of 2:1:3:3 (v/v).

In some aspects, the present disclosure comprises a combination of two or more active pharmaceutical ingredients (APIs). These combinations may further comprises one or more excipients. Some non-limiting examples of some excipients which may be used herein include a sugar or sugar derivative, such as mannitol, trehalose, or lactose, or an amino acid, such as glycine. These compositions may be dissolved in a solvent as described herein. In some embodiments, the active pharmaceutical ingredients (APIs) include but are not limited to an inhaled corticosteroid (ICS), a long acting β-agonist (LABA), or a long-acting muscarinic antagonist (LAMA). Some non-limiting examples of active pharmaceutical ingredients include salmeterol xinafoate, mometasome furoate, formoterol fumarate, tiotropium bromide, or budesonide. Such agents may be present in a ratio from about for the LABA to the ICS of from about 1:0.1 to about 1:100. In some embodiments, when the salmeterol xinafoate and mometasone furoate is used, then the ratio of the LABA to the ICS is from about 1:4.4. This ratio produces an amount of each component in an effective clinical dose.

In some aspects, the pharmaceutical composition comprises an excipient. In other aspects, the active pharmaceutical ingredient is formulated in the pharmaceutical composition without an excipient. When the composition comprises an excipient, the excipient may be present from about no excipient to a molar ratio of about 1:9 active pharmaceutical ingredients to the excipient. In some embodiments, the molar ratio of active pharmaceutical ingredients to excipients is from about a composition comprising no excipient to a molar ratio comprising about 1:1 ratio of active pharmaceutical ingredients to excipients. The molar ratio of active pharmaceutical ingredients to excipients may be about 1:1.

The composition may be dissolved in a solvent as described above. When the composition is dissolved in a solvent, the total amount of the pharmaceutical composition in the solvent may be from about 0.1% to about 10% (w/v). The total amount of the pharmaceutical composition may be from about 0.1% to about 6% (w/v). In some aspects, the total amount of the pharmaceutical composition is less than 6%, 5%, 4%, 3.5%, 3%, 2.5%, 2.0%, 1.75%, 1.5%, 1 1.25%, 1.0%, 0.9%, 0.8%, 0.7%, 0.6%, 0.5%, 0.4%, 0.3%, 0.2%, or 0.1%, or any range derivable therein. The total amount of the pharmaceutical composition in the solvent is preferable less than 6%, more preferably less than 5%. Using small amount of the pharmaceutical composition in the solvent is believed to give the advantageous properties such as leading to the formation of a brittle matrix particle and thus using less than 6% (w/v) and preferably less than 5% (w/v) is recommended. While lower amounts of the compounds are beneficial, the concentrations below 0.01% (w/v) or more preferably 0.1% (w/v) may lead to solutions too dilute to obtain a useful pharmaceutical composition. In some embodiments, the total amount of the pharmaceutical composition is about 0.5% (w/v).

In some aspects, the compositions are prepared using a thin film apparatus. The apparatus may be used to apply the solution to a surface such as a stainless steel and then frozen. This surface may also be rotating such that without wishing to be bound by any theory, it is believed that the rotating prompts the even application of the solution to the surface. The solution may be frozen at a cryogenic temperature such as a temperature below −50° C. Cryogenic temperatures include a temperature form about −50° C. to about −270° C., form about −70° C. to about −120° C., or form about −75° C. to about −100° C. In some embodiments, the cryogenic temperature is about 90° C.±3° C. In some aspects, the samples are stored frozen. In other aspects, the samples are lyophilized to obtain a dry powder. Lyophilization is known to those of skill in the art and is taught in U.S. Pat. Nos. 5,756,468, 6,440,101, 8,579,855, and PCT Patent Application Publication No. WO 2009/125986, which are incorporated herein by reference. In some aspects, it may be advantageous to store the composition at room temperature. The lyophilized samples may be prepared such that the temperature is gradually increased from the lyophilization temperature of less than −40° C. to a temperature around room temperature such as about 25° C. Also, he increase in temperature may be carried out under a vacuum or in a reduced pressure environment and/or an environment which has a reduced moisture content such as a desiccator.

The present disclosure provides brittle matrix particles which have a high surface area compared to other techniques such as jet milling or physical mixtures. In some aspects, the brittle matrix particles with two or more active pharmaceutical compositions have a specific surface area of greater than 5 m²/g. The brittle matrix particles may have a specific surface area from about 5 m²/g to about 1000 m²/g, from about 10 m²/g to about 500 m²/g, or from about 20 m²/g to about 250 m²/g. In some embodiments, the specific surface area is from about 5, 6, 7, 8, 9, 10, 15, 20, 25, 30, 35, 40, 45, 50, 60, 70, 80, 90, 100, 125, 150, 175, 200, 225, 250, 300, 350, 400, 450, 500, 600, 700, 800, 900, to about 1000 m²/g, or any range derivable therein. Methods of calculation the surface area of the composition are described herein in the Examples sections.

The brittle matrix particles comprising a pharmaceutical composition described herein may have a total emitted dose (or emitted dose) of greater than 80% of the active pharmaceutical ingredient. The total emitted dose may also be from about 80% to about 100%, from about 85% to about 100%, or from about 90% to about 100%. The formulations of the pharmaceutical composition described herein may have an total emitted dose of greater than 80%, 81%, 82%, 83%, 84%, 85%, 86%, 87%, 88%, 89%, 90%, 91%, 92%, 93%, 94%, 95%, 96%, 97%, 98% or 99%, or any range derivable therein. Methods of calculating the total emitted dose are described herein in the Examples section.

The pharmaceutical compositions described herein may comprise one or more excipients. Excipients are components which are not therapeutically active but may be used in the formation of a pharmaceutical composition. The excipients used herein include amino acids, sugars, sugar derivatives, or other excipients know those of skill in the art. In particular, the present disclosure includes the use of a sugar such as trehalose, lactose, glucose, fructose, or mannose, or a sugar derivative such as an aminosugar such as glucosamine or a sugar alcohol such as mannitol. Other excipients which may be used include amino acids such as alanine or glycine.

In some aspects, the brittle matrix particles component contains two or more drug molecules with one or more excipients to form a pharmaceutical composition. The pharmaceutical composition can thus be formulated in the brittle matrix particles in an amorphous form or in a particular crystalline form. In some embodiments, the pharmaceutical composition is formulated in the amorphous form. Additionally, the brittle matrix particles that may be used are a low density particle.

The present disclosure provides methods which makes use of the brittle matrix particles in the aerosol administration of a pharmaceutical composition. Without wishing to be bound by any theory, it is believed that the brittle matrix particles are readily fractured during the aerosolization thus enhancing the delivery of the pharmaceutical composition. The fracturing of the particles may be used to enhance the composition's ability to aerosolize and dispersion during administration.

In certain embodiments, the pharmaceutical compositions may be delivered by inhalation and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts and may be used in some embodiments. Transmucosal drug delivery in the form of a polytetrafluoroetheylene support matrix is described in U.S. Pat. No. 5,780,045 (specifically incorporated herein by reference in its entirety).

In some embodiments, a brittle matrix particle composition comprising active agents or drugs as described herein may be delivered intranasally, to the lungs, or via inhalation or aerosol delivery. In some embodiments the brittle matrix particle composition comprising active agents or drugs may be delivered via an inhaler (also called a puffer) or a metered dose inhaler. Inhaler apparatuses are well known in the art and may be used to for the delivery of drugs for the treatment of asthma or COPD.

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### II. ACTIVE PHARMACEUTICAL INGREDIENTS

In some aspects, the present disclosure provides pharmaceutical compositions formulated as a brittle matrix particle comprising two or more active pharmaceutical ingredients. These active pharmaceutical ingredients may be selected from a short acting β-agonist, a long acting β-agonist, a long acting muscarinic antagonist, or a corticosteroid. It is contemplated that any short acting β-agonist, a long acting β-agonist, a long acting muscarinic antagonist, or a corticosteroid may be used in the pharmaceutical compositions prepared herein. In some embodiments, the present disclosure provides compositions comprising either a long acting β-agonist and a corticosteroid or a composition with a long acting β-agonist, a long acting muscarinic antagonist, and a corticosteroid. These active pharmaceutical ingredients may be present in the pharmaceutical compositions described herein according to the ratios described in Table 1.

TABLE 1
Dosing and Ratio of Composition of Active Pharmaceutical
Ingredient in the Compositions Described Herein

| Active Pharmaceutical | | |
|---|---|---|
| Ingredient (API) | Ratio | Dosing |
| Long Acting β-agonist | 1 | about 1 to about 500 μg |
| Long Acting Muscarinic | about 0.1 to | about 1 to about 100 μg |
| Antagonist | about 100 | |
| Corticosteroid | about 0.1 to | about 1 to about 1000 μg |
| | about 100 | |

In some aspects, the compositions used herein may comprise a ratio of the long acting β-agonist to the corticosteroid from about 1:0.1 to about 1:100. Additionally, the composition may be characterized by the ratio of the long acting β-agonist to the long acting muscarinic antagonist wherein the ratio is from about 1:0.1 to about 1:100. The compositions that may be used herein have a ratio of the long acting muscarinic antagonist to the corticosteroid from about 0.1:100 to about 100:0.1. When the ratios are measured for a triple therapy, the composition may comprise a ratio of the long acting β-agonist, the long acting muscarinic antagonist, and the corticosteroid from about 1:0.1:0.1 to about 1:100:100. Non-limiting example of the ratio when used in dual therapy or a triple therapy include 1:4.4 or 1:2:35.5. In some aspects, the pharmaceutical compositions comprise a weight percentage of the active pharmaceutical ingredients in the composition from about 10 wt % to about 100 wt %.

These pharmaceutical compositions may also be formulated as a unit dose with a dose of the long acting β-agonist from about 1 μg to about 500 μg and a dose of the corticosteroid from about 1 μg to about 1,000 μg. In some embodiments, the pharmaceutical composition may further comprise a dose of the long acting muscarinic antagonist from about 1 μg to about 100 μg,

In some aspects, it is contemplated that any long acting β-agonist may be used in the formulations described herein. Some non-limiting examples of long acting β-agonist include formoterol fumarate, salmeterol xinafoate, bambuterol, clenbuterol, indacaterol, arformoterol, carmoterol, GSK-159797, GSK-597901, GSK-159802, GSK-642444, GSK-678007, or other long acting β-agonist known in the art. The long acting β-agonist may be formulated in a dose from about 1 μg, 5 μg, 10 μg, 20 μg, 40 μg, 60 μg, 80 μg, 100 μg, 120 μg, 140 μg, 160 μg, 180 μg, 200 μg, 225 μg, 250 μg, 275 μg, 300 μg, 325 μg, 350 μg, 400 μg, 450 μg, or 500 μg or any range derivable therein.

In some aspects, it is contemplated that any long acting muscarinic antagonist may be used in the formulations described herein. Some non-limiting examples of long acting muscarinic antagonist include salts of tiotropium, aclidinium, dexpirronium, ipratropium, oxitropium, darotropium, glycopyrronium, or glycopyrrolate derivative or other long acting muscarinic antagonist known in the art such as those taught by US Patent Application No. 2009/0181935, PCT Patent Application No. WO 2010/007561, and PCT Patent Application No. WO 2008/035157, which are incorporated herein by reference. The long acting muscarinic antagonist may be formulated in a dose from about 1 μg, 5 μg, 10 μg, 20 μg, 30 μg, 40 μg, 50 μg, 60 μg, 70 μg, 80 μg, 90 μg, or 100 μg, or any range derivable therein.

In some aspects, it is contemplated that any corticosteroid may be used in the formulations described herein. Some non-limiting examples of corticosteroid include beclomethasone dipropionate, budesonide, flunisolide, fluticasone propionate, mometasone furoate, ciclesonide, rofleponide palmitate, triamcinolone acetonide, or other corticosteroid known in the art. The corticosteroid may be formulated in a dose from about 1 μg, 5 μg, 10 μg, μg, 50 μg, 75 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, or 1,000 μg or any range derivable therein. The specific dose of each active pharmaceutical ingredient will vary depending on the specific compound present in the pharmaceutical composition.

In some aspects, it is contemplated that each of the active pharmaceutical ingredients could be formulated individually as separate pharmaceutical composition.

### III. RESPIRATORY INDICATIONS

In some aspects, the compositions of the present disclosure may be used in the treatment of respiratory diseases and conditions. Some non-limiting examples of respiratory disease and conditions include asthma, acute respiratory distress syndrome (ARDS), chronic pulmonary inflammatory disease (COPD), reactive airways dysfunction syndrome (RADS), airway hyperreactivity, eosinophilic or interstitial lung disease such as sarcoidosis or eosinophilic granulomatosis, chemical-induced lung injury, plastic bronchitis, bronchitis, chronic bronchitis, chronic obstructive pulmonary (airway) disease, silicosis, inhalational smoke induced acute lung injury (ISALI), or immune diseases and conditions such as allergic rhinitis and chronic sinusitis. The pharmaceutical compositions described herein may be used for the treatment of respiratory conditions such as asthma and chronic obstructive pulmonary disease and other obstructive airways diseases.

Chronic obstructive pulmonary disease (COPD) is a disease which is characterized by clinically poor airflow such as reduced which does not improve over several months. Cigarette smoking including long term cigarette smoking is believed to be the leading cause of COPD. Airflow obstruction in COPD is usually progressive in patients who continue to smoke eventually leading to disability and shortened survival time. Smoking cessation has been shown to slow the rate of decline to that of a non-smoker but the damage caused by smoking is irreversible. Other etiological factors (e.g., airway hyper responsiveness or hypersensitivity), air pollution (e.g., sulfur dioxide and possibly second hand smoke), occupational chemicals (e.g., cadmium) and generally allergy) have been identified in the literature but are believed to account for only a minority of COPD cases. Other risk factors include: heredity, second-hand smoke, exposure to air pollution at work and in the environment, and a history of childhood respiratory infections.

Some non-limiting examples of COPD include chronic coughing, frequent chest tightness, shortness of breath, an increased effort to breathe, increased mucus production, and frequent clearing of the throat. In some instances of COPD, airway obstruction is incompletely reversible, but other COPD patients do show some improvement in airway obstruction with treatment. Airway obstruction due to chronic and excessive secretion of abnormal airway mucus, inflammation, bronchospasm, and infection are believed to cause chronic bronchitis leading to chronic cough, mucus production or both. In emphysema instead, the elastin in the terminal bronchioles is destroyed leading to the collapse of the airway walls and inability to exhale. Emphysema is characterized by the destruction of the alveoli and the abnormal permanent enlargement of the air spaces distal to the terminal bronchioles, accompanied by destruction of their walls without apparent fibrosis.

Asthma is a chronic respiratory disease associated with increased inflammation particular in the airways. Some non-limiting examples of asthma symptoms include wheezing, coughing, chest tightness, shortness of breath, and increased difficulty in breathing. Asthma is generally not considered a type of COPD as asthma is generally reversible. The cough may result in the production of sputum including sputum with high levels of eosinophil. The frequency and severity of asthma symptoms may be greatly increased in the presence of triggers. Some non-limiting examples of asthma triggers include exercise, dust, pollution, pet dander, and other irritants and allergens. Asthma is generally diagnosed by observation of the pattern of symptoms, response to treatment, and spirometry. In some embodiments, there are several specific subtypes of asthma such as occupational, cough-variant, exercise-induced, aspirin-induced, or alcohol induced asthma. Another non-limiting example of a sub-type of asthma is brittle asthma which is characterized by recurrent and severe attacks. Furthermore, asthma may be associated or have its severity increased by other respiratory conditions such as bronchitis or allergies.

Clinical classification of asthma is assigned based upon the frequency of symptoms, the forced expiratory volume in one second (FEV₁), and peak expiratory flow rate or the asthma may be classified by its origin (atopic or extrinsic and topic or intrinsic). Atopic or extrinsic asthma typically is precipitated by allergens or other external factors, while topic or intrinsic asthma is not affected by allergens or other external factors. Additionally, asthma classifications based upon frequency or severity of symptoms. The clinical classifications include intermittent, mild persistent, moderate persistent, and severe persistent. For the intermittent classification, the patient generally experiences less than 2 incidents of symptoms per week, less than 2 nighttime incidents of symptoms per month, a FEV₁ of greater than or equal to 80%, with less than 20% variability, or require the use of a short acting β agonist two or less times per day. For the mild persistent classification, the patient generally experiences less than 2 incidents of symptoms per week, 3 to 4 nighttime incidents of symptoms per month, a FEV₁ of greater than or equal to 80%, with 20-30% variability, or require the use of a short acting β agonist more than two times per day. For the moderate persistent classification, the patient experiences symptoms daily, more than 1 nighttime experiences of symptoms per week, a FEV₁ from about 60% to about 80%, with greater than 30% variability, or require the use of a short acting β agonist daily. For the severe persistent classification, the patient experiences symptoms continuously, frequent nighttime experiences of symptoms (usually greater than 7 times per week), a FEV₁ of less than 60%, with greater than 30% variability, or require the use of a short acting β agonist more than twice daily.

Furthermore, asthma attacks or asthma exacerbations are classified as mild, moderate, or severe based upon the peak expiratory flow rate. For a mild attack, the peak expiratory flow rate is greater than 50% of the predicted best or greater than 200 L/min. For a moderate attack, the peak expiratory flow rate is between 25% and 50% of the predicted best or between 80 and 200 L/min. For a severe attack, the peak expiratory flow rate is less than 25% of predicted best or less than 80 L/min. Additionally, severe attacks may also have one or more additional symptoms or conditions. Severe attacks may be further sub-divided into three categories: acute, life-threatening, or near-fatal. For acute severe attacks, a patient may experience a respiratory rate greater than 25 breaths per minute, a heart rate of greater than 110 beats per minute, or be unable to speak a complete sentence in one breath. For life-threating severe attacks, a patient may experience altered levels of consciousness, exhaustion, arrhythmia, low blood pressure, cyanosis, a silent chest, poor respiratory effect, oxygen saturation of less than 92%, a PaO₂ of less than 8 kPa, or a normal to elevated PaCO₂. For near fatal severe attacks, a patient may experience either a high or elevated PaCO₂ or requires manual ventilation.

Asthma, chronic obstructive pulmonary disease and other respiratory diseases and disorders may be treated with β adrenergic receptor agonists as those compounds are known to provide a bronchodilator effect to the patients, resulting in relief from the symptoms of breathlessness. While β agonists may be used to provide symptomatic relief of bronchoconstriction in patients, another component of asthma or chronic obstructive pulmonary disease is inflammation, which often necessitates separate treatment. One non-limiting method of treating the inflammation caused by these respiratory diseases is through the use of steroids or corticosteroids. In some aspects, the administration of combinations of steroids, p adrenergic receptor agonists, and muscarinic antagonists in a single composition has shown an enhanced, synergistic, effect in terms of treatment of bronchoconstriction, inflammation and mucous secretions of airways in respiratory disease such as asthma and chronic obstructive pulmonary disease.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Methods

Two different combination therapies were produced by TFF, a cryogenic particle engineering technology (Yang et al., 2012), and characterized for their aerodynamic properties. The first, a dual combination therapy of salmeterol xinafoate (SX) and mometsone furoate (MF) was produced in a 5:22 SX to MF mass ratio. BMP formulations were produced as neat powders as well as with stabilizing excipients, lactose and mannitol, where drug loading totaled 50%. The second drug combination therapy produced by TFF was a triple combination of formoterol fumarate (FF), tiotropium bromide (TB), and budesonide (B) in a 1:2:35.5 FF:TB:B mass ratio. Mannitol was included as a stabilizing excipient so that the total drug loading was 50%. For comparison, in both dual and triple combinations, micronized drug was produced and blended with micronized excipient. Dry powder aerosols were generated from a HPMC capsule loaded into a HandiHaler® or Monodose® dry powder inhaler. Aerosols were characterized by Next Generation Pharmaceutical Impaction (NGI) operated at flows sufficient for a 4 kPa pressure drop across the device. This was equivalent to 51 L/min for the HandiHaler® and 90 L/min for the Monodose®.

### Example 2—Results

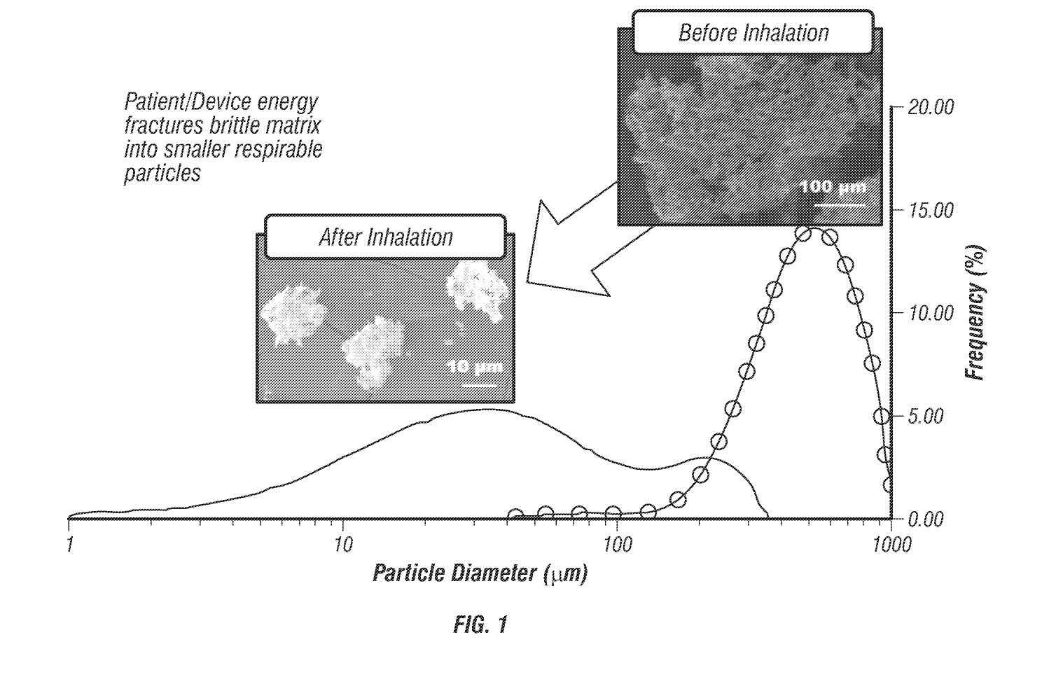

Particle engineering technologies, such as spray drying and TFF, allow for the formulation of multiple actives into a single inhalable particle. BMP created by TFF presents a paradigm shift in dry powder inhalation. As illustrated in FIG. 1, rather than using the inspiratory energy to deagglomerate discrete particles, this platform uses the energy generated by a patient/DPI for the brittle fracture of BMP into respirable low-density particles.

Since TFF is a bottom-up production method that begins with a solution, API and excipients are initially homogeneous at the molecular-level before application to a cryogenic surface for rapid freezing. Using this unique approach to DPI formulation, combinations therapies are homogenously dispersed within aerosol particles resulting in delivered dose uniformity and co-deposition of each API within a combination product. The data presented in Table 2 shows that the aerosol properties of SX and MF in BMP combination formulations are very similar for both APIs. Conversely, micronized powders of SX and MF delivered in a combination powder blend demonstrated different fine particle fractions (FPF) and mass median aerodynamic diameters (MMAD). Additionally, BMP SX/MF formulation without excipients (neat) was able to deliver nearly 50% of the both loaded drugs as fine particles.

TABLE 2
Aerodynamic properties of SX/MF combination BMP formulations and
Micronized formulations delivered by a Monodose ® inhaler.

| | FPF | FPF | MMAD | GSD | TED |
|---|---|---|---|---|---|
| Formulation | (% of deliv.) | (% of loaded) | (μm) | (μm) | (%) |
| Neat BMP SX/MF | | | | | |
| SX | 55.52 ± 4.78 | 52.90 ± 4.18 | 3.58 ± 0.32 | 3.55 ± 1.12 | 95.31 ± 1.13 |
| MF | 52.08 ± 4.40 | 48.91 ± 4.16 | 3.69 ± 0.26 | 3.33 ± 0.63 | 93.91 ± 1.27 |
| BMP Mannitol SX/MF | | | | | |
| SX | 44.60 ± 4.09 | 42.74 ± 3.22 | 4.45 ± 0.46 | 3.67 ± 1.29 | 95.92 ± 2.07 |
| MF | 43.91 ± 3.37 | 41.06 ± 3.47 | 4.43 ± 0.36 | 3.86 ± 1.53 | 93.49 ± 1.70 |
| Neat Micronized SX/MF | | | | | |
| SX | 59.51 ± 7.07 | 40.98 ± 6.77 | 3.30 ± 0.62 | 1.91 ± 0.06 | 68.59 ± 3.57 |
| MF | 34.29 ± 5.78 | 24.50 ± 4.91 | 4.45 ± 0.96 | 2.01 ± 0.02 | 71.18 ± 3.12 |
| Micronized Mannitol SX/MF | | | | | |
| SX | 57.52 ± 7.91 | 44.74 ± 6.82 | 3.41 ± 0.65 | 1.94 ± 0.07 | 77.67 ± 1.66 |
| MF | 30.07 ± 6.20 | 23.84 ± 5.05 | 4.84 ± 0.92 | 1.94 ± 0.02 | 79.20 ± 0.57 |
| Micronized Lactose SX/MF | | | | | |
| SX | 56.15 ± 9.58 | 41.39 ± 8.60 | 3.65 ± 0.58 | 1.00 ± 0.01 | 73.88 ± 1.14 |
| MF | 27.27 ± 6.01 | 21.09 ± 4.37 | 4.84 ± 0.81 | 1.90 ± 0.04 | 77.49 ± 3.46 |

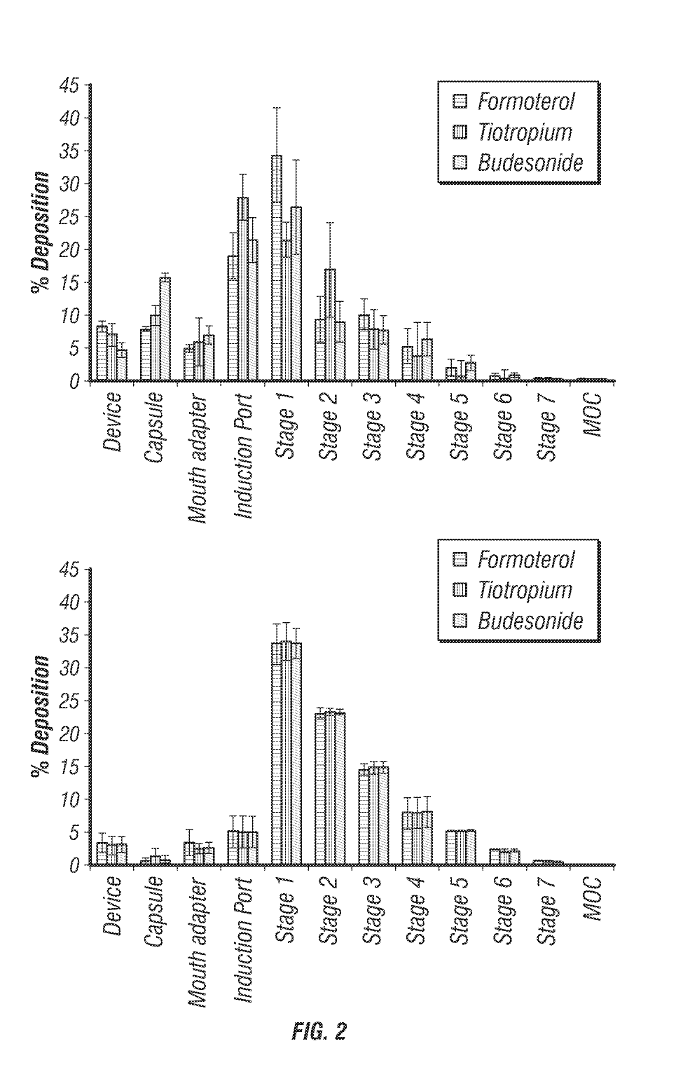

Assessment of the aerodynamic performance of a BMP triple combination of FF, TB and B produced similar results to the BMP SX/MF formulation. BMP formulation ensured that individual APIs were delivered and deposited in a homogenous manner. As evident in FIG. 2, the regional deposition of the BMP formulation was nearly identical for all three APIs. This BMP triple combination could improve therapy in patients where β2-agonists receptors are downregulated and long-acting muscarinic antagonist (LAMA) sensitivity is increased (Williamson et al., 2010). While FPF as a percentage of loaded dose was above 50% for all three BMP formulated APIs, further optimization of formulation/device is needed to reduce upper airway (stage 1 and 2) deposition in these formulations.

### Example 3—Materials and Methods

A. Material

Salmeterol xinafoate and mometasone furoate were purchased from Mascot I.E. CO., LTD (Changzhou, China). Alpha-lactose monohydrate and mannitol were purchased from Fisher Scientific (NJ, USA); D-Trehalose anhydrous was purchased from Acros Organics (NJ, USA) and glycine was purchased from J.T. Baker (PA, USA). High performance liquid chromatography (HPLC) grade acetonitrile and methanol were purchased from Fisher Scientific (NJ, USA). Water was purified by reverse osmosis (MilliQ, Millipore, France).

B. Formulation Preparation

Thin Film Freezing technology was used to produce BMP formulations for co-deposition. In brief, salmeterol xinafoate (SX), mometasone furoate (MF) and pharmaceutical excipients were dissolved in a co-solvent mixture of tertiary butanol, 1,4-dioxane, acetonitrile and purified water (2:1:3:3, v/v) (Jouyban-Gharamaleki et al., 2001. Combinations of a.) salmeterol xinafoate, mometasone furoate and lactose (SXMFLac), b.) salmeterol xinafoate, mometasone furoate and mannitol (SXMFMan), c.) salmeterol xinafoate, mometasone furoate and glycine (SXMFGly), d.) salmeterol xinafoate, mometasone furoate and trehalose (SXMFTre) and e.) salmeterol xinafoate, mometasone furoate (SXMF) without an excipient, were dissolved in the co-solvent solution. The ratio of salmeterol xinafoate to mometasone furoate was 50 to 220 by mass that corresponded to the clinical dose; while the ratio of APIs to excipient was controlled at a 1:1 molar ratio. Depending on the solubility, the total solid concentration of the co-solvent was 0.5% (w/v). The co-solvent solution was rapidly frozen on a cryogenically cooled (−90±3° C.) rotating stainless steel surface of the thin film apparatus. After removal by a scraper, the frozen films were then collected in a container filled with liquid nitrogen to maintain the frozen state. The frozen samples were transferred to a VirTis Advantage Lyophilizer (VirTis Company Inc., Gardiner, N.Y.) to obtain dry powders. The formulations were lyophilized over 24 h at −40° C. at a pressure less than 200 mTorr, and then the shelf temperature was gradually increased to 25° C. over another 24 h. The final product was stored in a vacuum desiccator at room temperature.

For comparison purposes, the micronized crystalline physical blends of SX, MF and excipients, in the same ratio as used in the BMP formulations, were prepared by jet milling. Single bulk material was fed into an air-jet mill (Aljet mill, Fluid Energy, Plumsteadville, Pa., USA) with a feed pressure of 80 psi and a grinding pressure of 65 psi. Each bulk material was processed until the particle size reached the respirable range (1-5 μm) (Watts et al., 2013), and this varied between 5-7 times. Samples were collected and analyzed from the collecting chamber. The particle size before and after milling was measured using a Sympatec Helos laser diffraction instrument (Sympatec GmbH, Germany) equipped with a R3 lens. Equivalent amounts of micronized SX, MF and excipients were accurately weighed and mixed separately using the geometric dilution technique, and then placed into a stainless steel mixing vessel, blended by Turbula Blender T2F (Bachofen, Switzerland) for 30 min at 48 revolutions per minute (rpm).

C. Differential Scanning Calorimetry

Thermal analysis of BMP SXMF, BMP SXMFLac, BMP SXMFMan, BMP SXMFGly, BMP SXMFTre and each of their micronized components were conducted using modulated temperature DSC (Model 2920, TA Instruments, New Castle, Del.) equipped with refrigerated cooling system, coupled with TA Universal Analysis 2000 Software (New Castle, Del.). Calibration of the DSC instrument was carried out using indium as a standard. Sample powders (5-10 mg) were loaded in an aluminum pan and press-sealed with an aluminum lid (PerkinElmer, Waltham, Mass.). A crimped empty pan was used as reference. The mass of each empty sample pan was matched with the mass of empty reference pan to ±0.1 mg. Melting endotherm was analyzed at a heating ramp rate of 10° C./min and modulation temperature amplitude of 1° C. in range of 30-350° C. under nitrogen gas flow rate of 40 mL/min through DSC cell. The glass transition temperature (T_{g}) of BMP formulations was measured by modulated DSC but at a heating ramp rate of 5° C./min.

D. Powder X-Ray Diffraction

The morphology of the powder samples was evaluated by wide angle XRD (Rigaku R-Axis Spider, Japan) with an image plate detector using a graphite monochromator with CuKα radiation (=1.5418 Å). An acceleration voltage of 40 kV and current of 40 mA was used. Samples were mounted on a Hampton Research CryoLoop. The 2-dimensional image plate data was converted to a conventional 1-dimensional powder pattern using Rigaku's 2DP Version 1.0 data conversion program. In this study the conversion range was from 2° to 40° at a 2θ step size of 0.010. The data were analyzed using Bruker Analytical's DiffracPLUS Evaluation Package, EVA (V. 2009). XRPD patterns were established with the freshly prepared BMP formulations and the micronized blends, as well as the micronized and BMP individual APIs.

E. Scanning Electron Microscopy

Scanning electron microscopy (SEM) was employed to evaluate the surface morphology of both micronized and BMP formulations. Prior to imaging, samples were loaded onto double sided carbon tape and coated with platinum/palladium targeted for 12 nm thickness using sputter coater 208 HR (Cressington Scientific Instruments, Watford, England). SEM images were captured using a SmartSEM® graphical user interface software in a Carl Zeiss Supra® 40VP (Carl Zeiss, Oberkochen, Germany) under high vacuum mode with an operating Electron High Tension (EHT) of 5 kV.

F. Specific Surface Area Analysis

Specific surface area (SSA), which is related to powder porosity, was measured using a Monosorb MS-22 rapid surface area analyzer (Quantachrome Instruments, Boynton Beach, Fla.) with 30% nitrogen in helium as the adsorbate gas. Powder samples were degassed in a Thermoflow™ Degasser for at least 2 hours at 30° C. using nitrogen prior to analysis. The Monosorb utilizes a modified BET equation for extremely rapid, single-point determinations of surface area (P/P0=0.294).

G. Fourier-Transform Infrared Spectroscopy

Infrared spectra were acquired using Nicolet™ iS™50 FT-IR Spectrometer (Thermo Scientific, Waltham, Mass.) equipped with the Polaris™ long-life IR source (Thermo Scientific, Waltham, Mass.) and a DLaTGS-KBr detector. Each sample was dispersed in KBr using a mortar and pestle, and then a pellet was formed by applying pressure.

FTIR measurements were performed in the transmittance mode. The scanning range was from 700 cm⁻¹ to 3600 cm⁻¹, with a resolution of 4 cm⁻¹, 16 scans were recorded for each spectrum and the spectra were corrected against the background spectrum. All measurements were performed at room temperature.

2.8. Water Sorption

Water sorption profiles were determined for the BMP formulations using Dynamic Vapor Sorption (Surface Measurement Systems Ltd, London, UK). Consideration of the capacity (0.5 mL) of glass sample cells, 3 to 6 mg samples were loaded depending on particle density. As an initial step in all experiments, water absorbed during environmental exposure in storage or loading was removed by holding the samples at 0% relative humidity (RH) for 480 min or until the mass changed by less than 0.002% in dm/dt for 40 min. Each formulation was run for a complete sorption/desorption cycle between 0 and 90% relative humidity (RH) in steps of 10% RH at 25° C. The instrument was run in dm/dt mode to decide when equilibrium was reached, as determined by a dm/dt less than 0.002% within an interval of 5 min. Sorption isotherms were calculated and plotted according to percent change in mass minus the initial dry formulation weight. The mass % sorption (desorption) was calculated by dividing the increase (decrease) in mass due to water sorption (desorption) by the mass of the dry BMP formulations after the initial equilibration at 0% RH, and multiplying by 100.

H. Aerodynamic Particle Size Analysis

A Next Generation Pharmaceutical Impactor (NGI) (MSP Corp., Shoreview, Minn.) was used to determine the influence of DPI devices and to compare aerodynamic properties of BMP formulations and crystalline micronized blends. Around 2 mg powders were manually filled into size 3 HPMC capsules (Capsugel, Peapack, N.J.) and aerosolized using a Miat monodose Inhaler® DPI device, which was attached to the induction port by a molded silicone mouthpiece adapter. To characterize the performance of BMP formulations and micronized powders in vitro for animal study, powders were also filled into the insufflator chamber without capsule. Aerosols were produced over 2.7 s at an air flow rate of 91 L/min for Miat monodose Inhaler® and 4 s at an air flow rate of 51 L/min for insufflator to achieve an inhalation volume of 4 L and a 4 kPa pressure drop across the device (Wang et al., 2014; Watts et al., 2013). Stage cut-off size diameters of both devices were calculated to be 6.48, 3.61, 2.30, 1.37, 0.76, 0.43, 0.26 and 0.26 μm stages 1 through 7 and micro-orifice collector (MOC) (Marple et al., 2003). Before each run, NGI collection surfaces were coated with 1% (v/v) polysorbate 80 in ethanol, which is a coating material recommended by the European Pharmaceutical Aerosol Group (EPAG), to avoid bias caused by particle bounce and reentrainment[41]. After aerosolization, all collection surfaces were rinsed with known volumes of mobile phase. Powders deposited in the capsule, device, adaptor, throat, pre-separator, and stages 1-MOC were extracted respectively. After passing through a 0.2 μm PTFE filter, the solutions were analyzed by HPLC for SX and MF content. A pre-separator was equipped for DPI devices, not for an insufflator. For each test, total emitted dose (TED) was defined as the percentage of API emitted from the inhaler with respect to the total loaded dose into the capsule. Geometric standard deviation (GSD) and mass median aerodynamic diameter (MMAD) were calculated according to the USP 32-NF 27 General Chapter 601 (DiNunzio et al., 2008), based on the dose deposited on stages 1-MOC. The fine particle fraction (FPF) was defined as the mass fraction of particles less than 5.0 μm with the emitted dose. A plot of cumulative percentage of mass less than stated aerodynamic diameter versus aerodynamic diameter (cut-off size for each stage) was built and fit to a 4 parameter logistic curve using Sigmaplot (Systat Software Inc., San Jose, Calif.).

I. Chromatographic Analysis

All rinsed surface collections from capsule, device, adaptor, throat, pre-separator and stages 1-MOC were filtered and analyzed by high performance liquid chromatography (HPLC). HPLC was used to quantify the collected SX and MF. Samples were analyzed using a Dionex 3000 high performance liquid chromatography (HPLC) system equipped with a Phenomenex® reversed phase C₁₈ column 5 μm 150 mm×4.60 mm. All tests were conducted at room temperature with a flow rate of 1.2 mL/min. The mobile phase consisted of 90/10 (v/v) methanol/water, pH 3.5 adjusted with phosphoric acid. The injection volume was 20 μL and the detection wavelength was 250 nm. Linearity test solutions were prepared from SX and MF stock solutions at seven-concentration levels ranging from 1 μg/mL to 250 μg/mL.

The SX is marketed as racemic mixture and no evidence indicates that the (S)-salmeterol has different effects compared to the corresponding (R)-salmeterol, thus chiral chromatographic analysis was employed to identify the chirality of BMP formulations. To determine the optical activity of SX in the inhaled powders, chiral chromatography was performed using a Dionex 3000 HPLC, and a Phenomenex® normal phase chiral column Lux Cellulose-4 (5 μm 250×4.60 mm). The stereochemical separation of (R)-salmeterol and (S)-salmeterol was accomplished using a mobile phase containing n-hexane/2-propanol (90:10, v/v) with 0.1% DEA. The analysis was carried out at ambient temperature using a flow rate 1.0 mL/min at 250 nm UV.

J. In Vivo Pharmacokinetic Studies

An animal study was approved by the University of Texas at Austin Institute of Animal Care and Use Committee (IACUC). Male and female Sprague Dawley rats with their jugular vein pre-catheterized and non-pre-catheterized (Charles River Laboratories International, Inc., Wilmington, Mass.) weighing between 250-300 g with an average weight of 270 g were used for the in vivo intubation and dry powder insufflation procedure. During one week of acclimation time, the rats were housed two per cage in a 12-h light/dark cycle with food and water available ad libitum. Catheters were flushed every 3 days before the study with 500 Units/mL heparinized normal saline. Animals were divided into two groups, and each group contained 12 non pre-catheterized rats for lung sample collection and 4 pre-catheterized rats for blood sample collection.

A DP-4M Dry Insufflator™ device and AP-1 air pump and LS-2 small animal laryngoscope were used (Penn-Century, Inc., Wyndmoor, Pa.). BMP and micronized blends were weighed and loaded into the chamber of the insufflator (target SX dose=450 μg/kg, target MF dose=1980 μg/kg) before being attached to the adjustable-volume air pump. Each rat was anesthetized intraperitoneally with a 0.2 micron filtered mixture of ketamine HCl/xylazine HCl (80 mg/kg ketamine HCl, 6 mg/kg xylazine HCl), about 0.2-0.3 mL of the mixture was administered based on weight. After the rat was anesthetized, it was placed on its back at a 45-degree angle, and the incisors were secured with a small rubber band. The laryngoscope was used to visualize the trachea, then the insufflator device was inserted into the trachea and the air pump was attached to the insufflator device. The desired volume (2 mL of air per pump for rats) of air was dispensed from the attached air pump to force the powder from the chamber of the insufflator device through the PE tube, the metal-tipped cannula, and into the trachea of the rats (Morello et al., 2009). All powders were actuated into the lungs using 4-7 pumps. The insufflator was weighed before and after powder filling and administration, to confirm the actual dose insufflated by mass.

Following insufflation, blood samples were withdrawn from a jugular vein catheter at time points of 5 min, 15 min, 30 min, 1 h, 2 h, 4 h, 8 h, 12 h and 24 h followed by injecting the equal volume of warm normal saline back into the rats and stored in a BD Vacutainer® blood collection tube. In each group, three rats were sacrificed at each time point (15 min, 1 h, 2 h, 6 h and 24 hours) for lung harvest. To investigate the amount of API deposited in the lower respiratory tract, a modified bronchoalveolar lavage (BAL) procedure was performed by washing respiratory tract twice with 3 mL sterile phosphate buffered saline (PBS) for collection of fluids prior to lung sample collection (Chougule et al., 2007). Approximately 4-5 mL BAL was yielded. All bio samples were stored in −80° C. freezer until analyzed.

K. Quantification of SX and MF Concentrations in the Lung Tissue, BAL and Blood

SX and MF concentrations in the lung tissue, BAL and plasma were quantified by liquid chromatography mass spectrometry (LC/MS/MS). Lorcaserin (LOR) was employed as the internal standard. The LC/MS/MS system consisted of a Shimadzu CBM-20A Controller, two LC-20AD pumps, SIL-20AC autosampler, CTO-20AC column oven, and an AB Sciex API 4000-QTrap mass spectrometer with turbo ion spray. Chromatographic separation was achieved with an Ace Excel 3 Super C18 (3×75 mm, 3μ) purchased from MacMod (Chadds Ford, Pa.) and was maintained at 40° C. during the chromatographic runs. Mobile phase A contained 0.1% formic acid in Millipore H₂O and mobile phase B contained 0.1% formic acid in acetonitrile. The flow rate of the mobile phase was 0.7 mL/min. SX and MF were eluted with a step gradient. The column was equilibrated with 60% mobile phase B. At 2.5 minutes after injection, the system was switched to 95% mobile phase B. At 6 min, the system was switched back to 60% mobile phase B in preparation for the next injection. The following Q1/Q3 transitions were monitored: m/z 416.1→232.0 for SX, m/z 521.9→355.2 for MF, and m/z 196.0→129.1 for LOR (IS). The underlined transitions were used for quantification.

Quantification of SX and MF in plasma: SX/MF was quantified in plasma (EDTA). Briefly, 100 μL of calibrator and unknown plasma samples were mixed with 10 μL of LOR working stock solution (internal standard) and 300 μL of Mobile Phase B (0.1% formic acid in acetonitrile). The samples were vortexed vigorously for 2 min, and then centrifuged at 3200 g for 20 min at 23° C. Supernatant was transferred to 1.5 mL microfilterfuge tubes, centrifuged, and then 50 μL of the final extracts were injected into the LC/MS/MS. The ratio of the peak area of SX and MF to that of the internal standard LOR (response ratio) for each unknown sample was compared against a linear regression of calibrator response ratios at 0, 0.5, 1, 10, 50, 100, 500, 1000 ng/mL to quantify SX and MF. The concentration of each analyte was expressed as ng/mL plasma.

Quantification of SX and MF in lung tissue: the lung's weight was 1.6 grams on average. Briefly, calibrator, control, and unknown tissue samples were mixed with a 10 volumes of mobile phase B and homogenized with a tissue homogenizer. Samples were centrifuged at 3200 g for 20 min and then 100 μL of supernatant were transferred to 1.5 mL microfilterfuge tubes and spiked with 10 μL of LOR working stock solution. The samples were vortexed vigorously for 2 min, transferred to 1.5 mL microfilterfuge tubes, centrifuged, and then 10 μL of the final extracts were injected into the LC/MS/MS. The ratio of the peak area of SX and MF to that of the internal standard lorcaserin (response ratio) for each unknown sample was compared against a linear regression of calibrator response ratios at 0, 10, 100, 1000, 2500, 5000, 7500, 10000 ng/mL to quantify SX and MF. The concentration of SX and MF was normalized to protein content and expressed as μg/g of tissue (parts per million).

Quantification of SX and MF in BAL: 100 μL of calibrator, control, and unknown samples were mixed by vortexing with 300 μL of Mobile Phase B. After vortexing the samples were transferred to 1.5 ml microfilterfuge tubes and then centrifuged at 3200 g for 15 min at 23° C. Filtrates were transferred to autosampler tubes and 10 μL of the final extracts were injected into the LC/MS/MS. The peak area of SX and MF for each unknown sample was compared against a linear regression of calibrator peak areas at 0, 0.5, 1.0, 5, 10, 50, 100, 1000, 5000, 10000 ng/mL to quantify SX and MF. The concentration of SX and MF was expressed as ng/mL BAL.

### Example 4—Results for SX and MF Combination

A. Differential Scanning Calorimetry

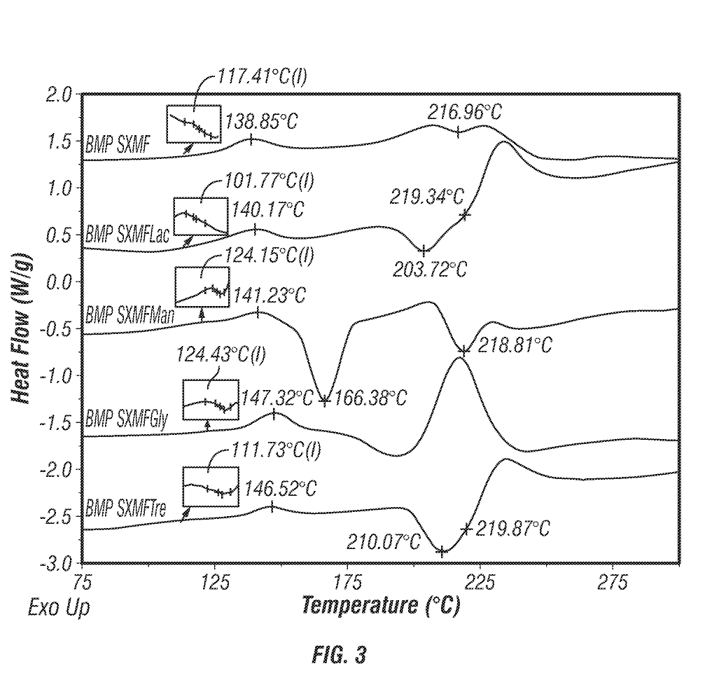

The results shown in FIG. 3 indicate that BMP formulations exhibited no melting endotherms, however a single glass transition (T_{g}) in the temperature range of 101.77° C. to 124.43° C. was observed by modulated DSC, which was followed by a single recrystallization peak in the temperature range of 138.85° C. to 147.32° C., respectively.

B. X-Ray Powder Diffraction

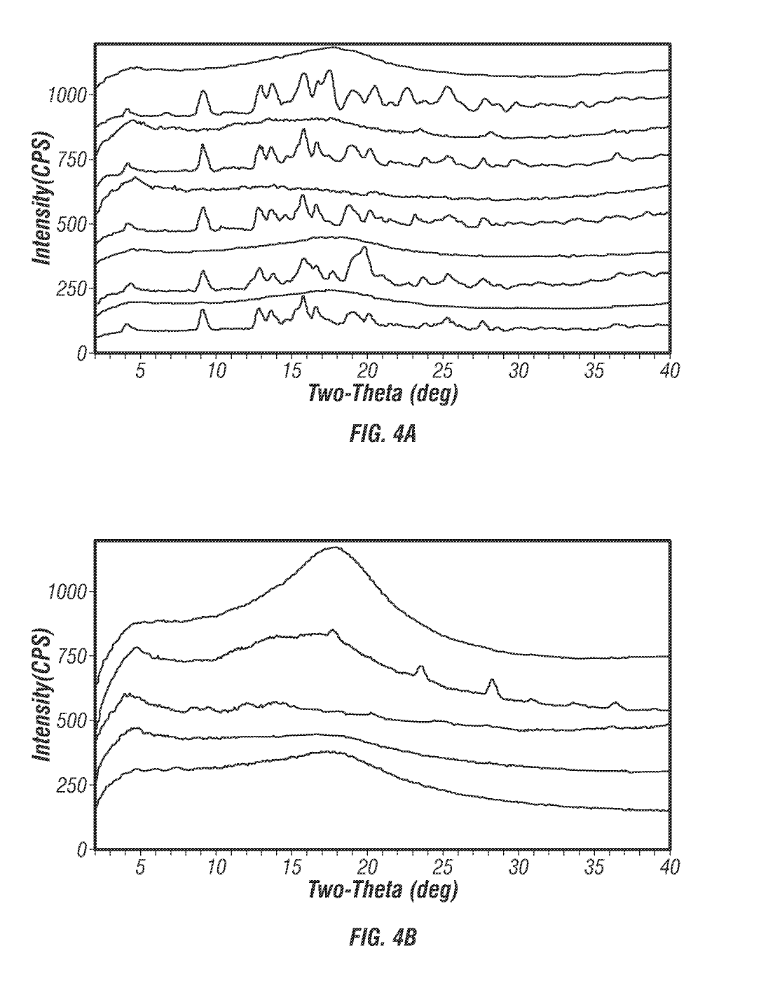

Absence of crystallinity was confirmed in FIG. 4A by the presence of a halo pattern in the diffractogram for the fixed dose combinations BMP SXMF, BMP SXMFLac and BMP SXMFTre after lyophilization. However, excipients in the fixed dose combination drug mixtures exhibited different crystalline properties as evidenced by XRPD. Gly and Man peaks were observed for BMP SXMFGly and BMP SXMFMan. The physical blends of the micronized drugs exhibited characteristic crystal peaks for SX and MF, respectively.

Stability results shown in FIG. 4B conducted on the fixed dose combinations prepared by TFF at 25° C./30% RH for 6 months exhibited no changes in XRPD peak intensity of BMP SXMF and BMP SXMFTre over 6 months, and the powders remained amorphous. In contrast, XRPD diffraction patterns showed a small crystalline peak corresponding to the SX (4.21 2θ) was observed for BMP SXMFLac, BMP SXMFMan and BMP SXMFGly over the same time period.

C. Scanning Electron Microscopy and Specific Surface Area Analysis

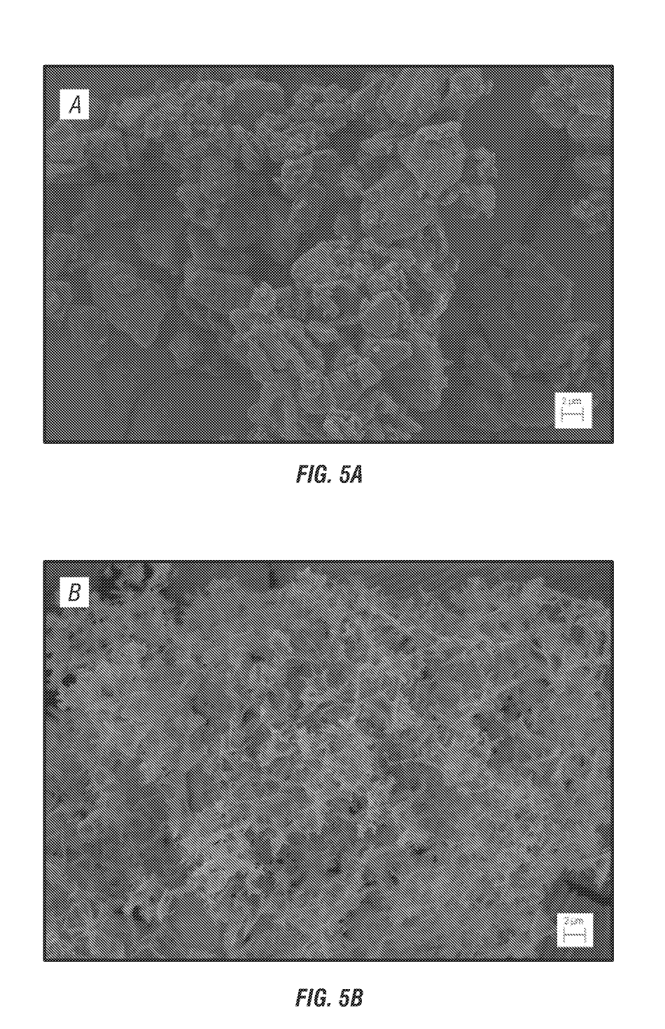

As exemplified in FIGS. 5A & 5B, the BMP fixed dose combinations exhibited porous microparticulate aggregates loosely connected to form a sponge-like structure with adjacent aggregates, which appeared homogeneous and brittle. In contrast, micronized drug physical blends were not homogeneous.

The results of BET surface area measurements are shown in Table 3. The specific surface area of micronized SXMF was found to be 0.89 m²/g. Generally, the specific surface area of BMP fixed dose combinations for co-deposition varied from around 26.87 to 35.61 m²/g. BMP SXMFLac and BMP SXMFTre showed larger surface areas as compared to the other BMP formulations. However, there was no significant difference between the values of BMP formulations.

TABLE 3
Specific surface area (SSA) of micronized SXMF, BMP SXMF, BMP
SXMFLac, BMP SXMFMan, BMP SXMFGly and BMP SXMFTre.
Values are expressed as mean ± SD (n = 3).

| Formulation | SSA (m²/g) |
|---|---|
| Micronized SXMF | 0.89 ± 0.01 |
| BMP SXMF | 28.18 ± 1.038 |
| BMP SXMFLac | 35.61 ± 1.682 |
| BMP SXMFMan | 29.87 ± 0.588 |
| BMP SXMFGly | 26.87 ± 1.255 |
| BMP SXMFTre | 32.41 ± 0.456 |

D. Fourier Transform Infrared Spectroscopy

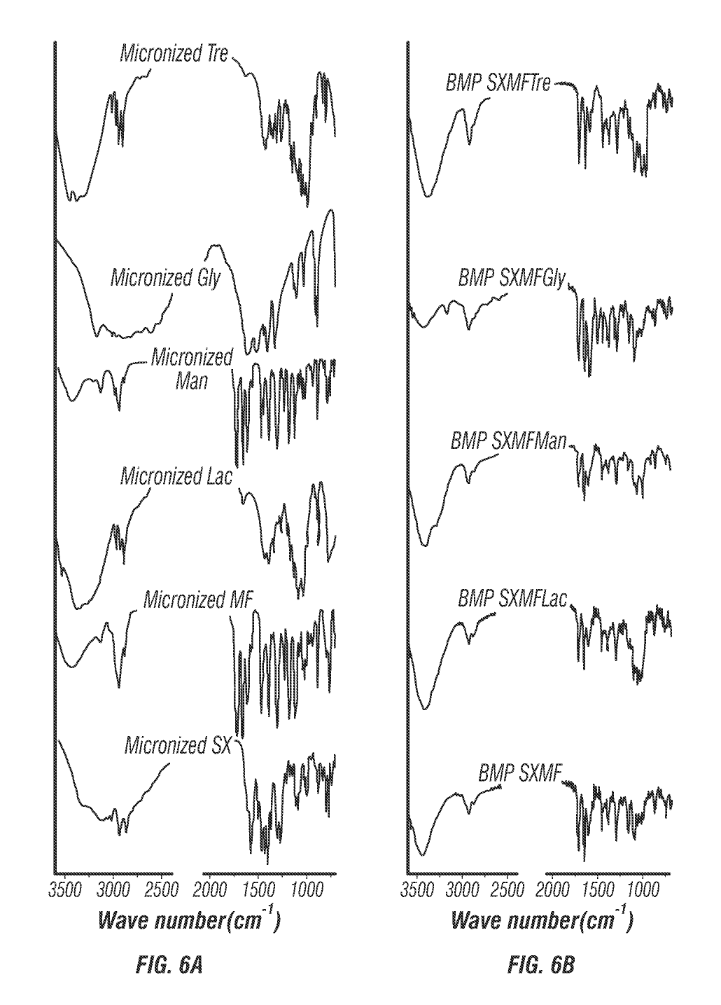

As shown in FIGS. 6A & 6B, Fourier transform infrared spectroscopy (FTIR) spectrum for individual components and TFF processed powders were investigated and examined. The peak of —C═O stretching at 1724 cm⁻¹ could be observed for all BMP, also including the single SX and MF. The strong peak located in the region of 3200 cm⁻¹ to 3700 cm⁻¹ could correspond to the —OH or —NH, which was observed in all single materials. Single SX showed strong peaks at 3278 cm⁻¹ (shown in FIG. 6A), which was attributed to —NH stretching of the secondary amide. The —C—N stretching for SX was confirmed by presence of relatively sharp peak at 1268 cm⁻¹. In the carbonyl region, the individual crystalline forms exhibited broad peaks compared to fixed dose combinations. No band shift was observed at the —C═O stretching peak at 1724 cm⁻¹ in all fixed dose combinations as compared to crystalline MF. Strong and wide —NH and —OH peaks in the region of 3200 cm⁻¹ and 3400 cm⁻¹ were seen for the single materials, especially for the excipients. Overall, the wave numbers of either —C═O or —NH and —OH did not shift for fixed dose combinations after TFF process. This lack of shifting indicates no evidence of H-bonding.

E. Water Sorption

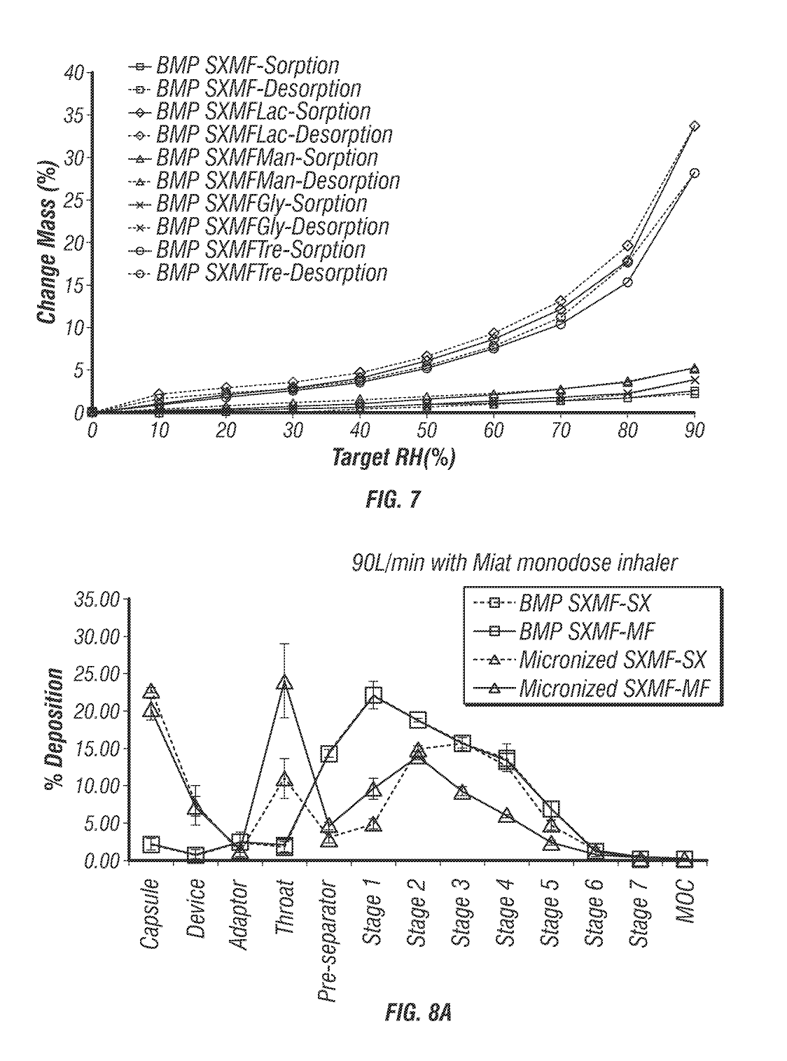

FIG. 7 showed the results of BMP fixed dose combinations for the sorption and desorption isotherms determined after one cycle by DVS. Slow moisture uptake was observed from BMP SXMFLac and BMP SXMFTre during sorption testing. The time required to a complete the sorption/desorption cycle was 22 h and 19 h, respectively, since slow water absorption (and corresponding slow baseline stabilization) meant long cycle time compared to BMP SXMFMan (12 h), BMP SXMFGly (11.5 h) and BMP SXMF (11 h). The moisture uptake as measured by weight gain (%) was BMP SXMFLac (˜35%), BMP SXMFTre (˜30%), BMP SXMFMan (˜5%), BMP SXMFGly (˜4%) and BMP SXMF (˜2%). This suggests that the BMP SXMFLac and BMP SXMFTre were more hydroscopic than the other compositions.

F. In Vitro Aerosol Performance

The NGI dispersion data for the fixed dose BMP formulations and micronized API blends were plotted and shown in Table 4. The aerosol properties of all fixed dose combinations administered with the Miat monodose Inhaler® revealed high FPF (38%-57%) and small MMAD (around 4 μm).

TABLE 4
Aerodynamic properties of SX and MF for co-drug deposition BMP and crystalline micronized powders
under 90 L/min with Miat monodose inhaler ®. Values are expressed as mean ± SD (n = 3).

| | | FPF | FPF | MMAD | GSD | TED |
|---|---|---|---|---|---|---|
| Formulation | | (% of deliv.) | (% of loaded) | (μm) | (μm) | (%) |
| BMP SXMF | SX | 55.52 ± 4.78 | 52.90 ± 4.18 | 3.58 ± 0.32 | 3.55 ± 1.12 | 95.31 ± 1.13 |
| | MF | 52.08 ± 4.40 | 48.91 ± 4.16 | 3.69 ± 0.26 | 3.33 ± 0.63 | 93.91 ± 1.27 |
| BMP SXMFLac | SX | 42.13 ± 1.46 | 40.31 ± 0.76 | 3.68 ± 0.22 | 2.39 ± 1.21 | 95.72 ± 2.28 |
| | MF | 40.90 ± 1.26 | 38.64 ± 0.97 | 3.97 ± 0.35 | 3.56 ± 0.60 | 94.50 ± 1.64 |
| BMP SXMFMan | SX | 44.60 ± 4.09 | 42.74 ± 3.22 | 4.45 ± 0.46 | 3.67 ± 1.29 | 95.92 ± 2.07 |
| | MF | 43.91 ± 3.37 | 41.06 ± 3.47 | 4.43 ± 0.36 | 3.86 ± 1.53 | 93.49 ± 1.70 |
| BMP SXMFGly | SX | 56.45 ± 7.38 | 53.57 ± 5.87 | 3.62 ± 0.75 | 2.66 ± 0.36 | 95.14 ± 4.27 |
| | MF | 53.38 ± 6.55 | 49.98 ± 5.02 | 3.74 ± 0.68 | 2.58 ± 0.25 | 93.86 ± 3.99 |
| BMP SXMFTre | SX | 42.18 ± 3.37 | 39.76 ± 1.27 | 4.09 ± 0.57 | 2.64 ± 0.55 | 94.55 ± 5.43 |
| | MF | 37.85 ± 5.74 | 35.93 ± 4.77 | 4.10 ± 0.48 | 2.61 ± 0.46 | 95.13 ± 3.24 |
| Micronized SXMF | SX | 59.51 ± 7.07 | 40.98 ± 6.77 | 3.30 ± 0.62 | 1.91 ± 0.06 | 68.59 ± 3.57 |
| | MF | 34.29 ± 5.78 | 24.50 ± 4.91 | 4.45 ± 0.96 | 2.01 ± 0.02 | 71.18 ± 3.12 |
| Micronized SXMFLac | SX | 56.15 ± 9.58 | 41.39 ± 8.60 | 3.65 ± 0.58 | 1.00 ± 0.01 | 73.88 ± 1.14 |
| | MF | 27.27 ± 6.01 | 21.09 ± 4.37 | 4.84 ± 0.81 | 1.90 ± 0.04 | 77.49 ± 3.46 |
| Micronized SXMFMan | SX | 57.52 ± 7.91 | 44.74 ± 6.82 | 3.41 ± 0.65 | 1.94 ± 0.07 | 77.67 ± 1.66 |
| | MF | 30.07 ± 6.20 | 23.84 ± 5.05 | 4.84 ± 0.92 | 1.94 ± 0.02 | 79.20 ± 0.57 |
| Micronized SXMFGly | SX | 59.70 ± 9.63 | 47.96 ± 7.58 | 3.39 ± 0.67 | 1.88 ± 0.09 | 80.37 ± 0.34 |
| | MF | 32.73 ± 8.49 | 26.58 ± 5.84 | 4.71 ± 1.02 | 1.89 ± 0.06 | 81.86 ± 3.86 |
| Micronized SXMFTre | SX | 60.23 ± 3.50 | 47.11 ± 2.71 | 2.91 ± 0.29 | 1.91 ± 0.05 | 78.55 ± 8.66 |
| | MF | 19.81 ± 5.86 | 16.54 ± 7.14 | 4.29 ± 0.37 | 1.92 ± 0.06 | 81.54 ± 9.34 |

However, the aerosol properties of SX corresponded with MF for fixed dose BMP formulations. The FPF of micronized blend SXMF was 59.51±7.07% for SX versus 34.29±5.78% for MF, while the FPF of BMP SXMF was 55.52±4.78% for SX versus 52.08±4.40% for MF, which were similar. Surprisingly, the MMADs for both formulations displayed similar values loading in the range of 3-5 μm. However, the MMAD of two APIs were close for fixed dose combination. Conversely, the micronized blends of SX and MF exhibited different MMADs. In addition, TEDs of two APIs for BMP formulations were approximately above 93% and the TED for micronized blends was below 82%.

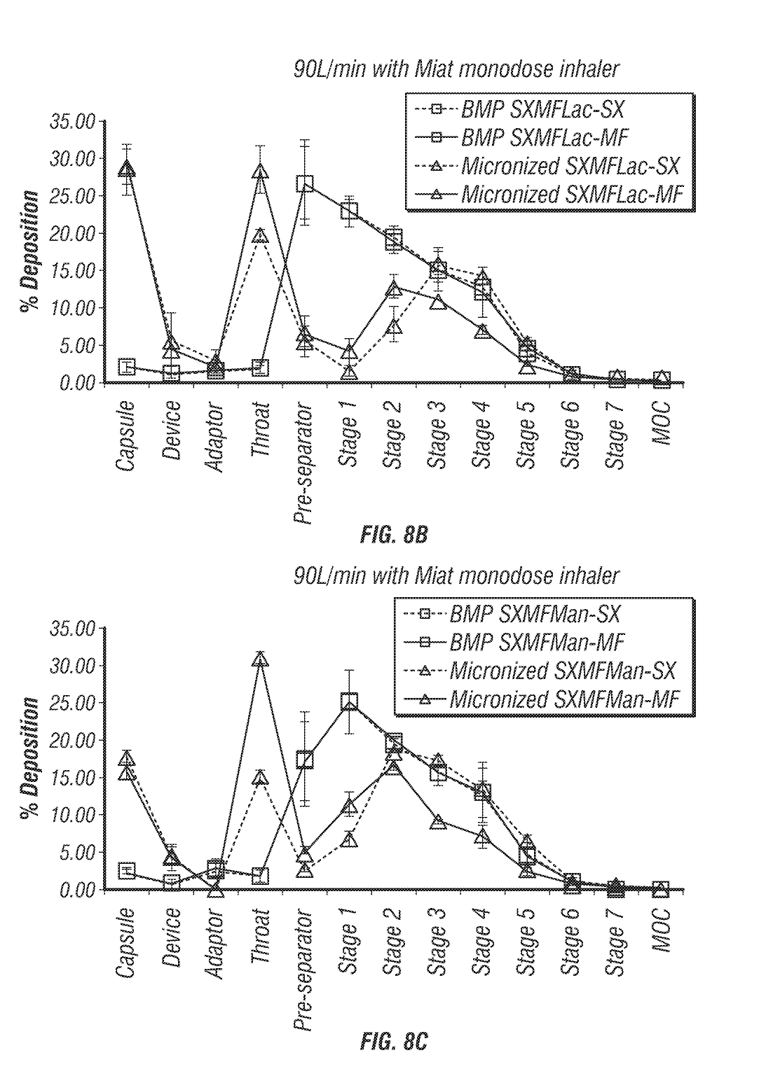

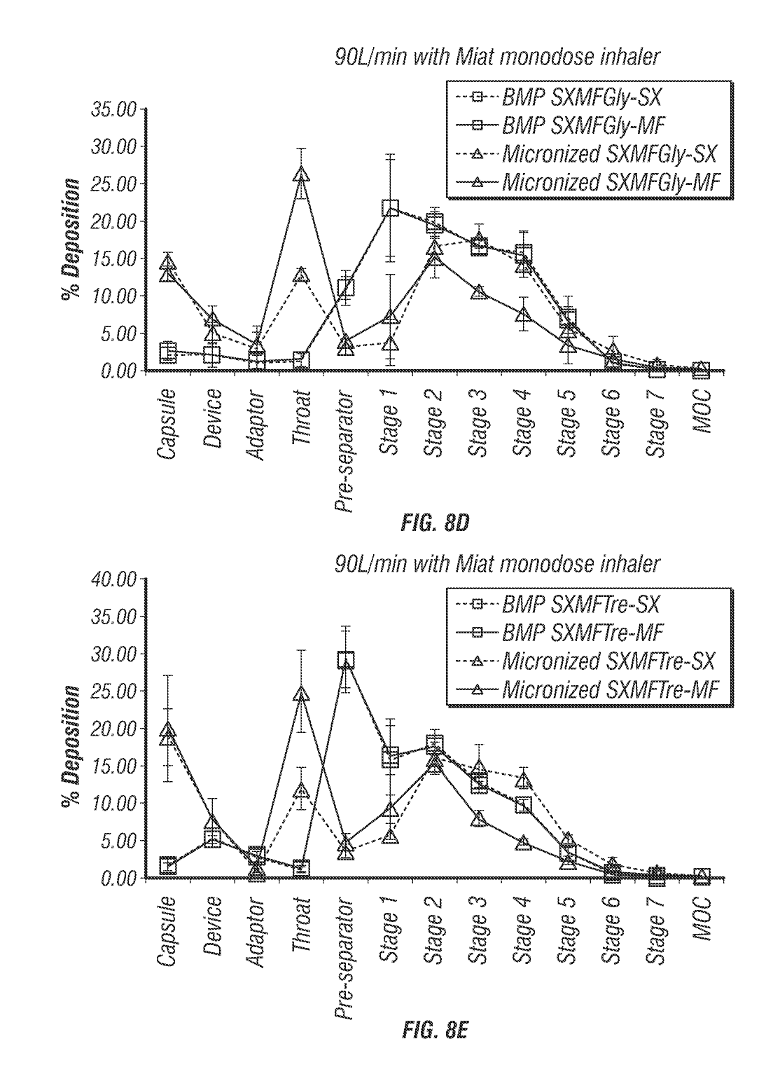

Inspection of the NGI data in FIGS. 8A-8E showed that, the deposition percentages in different stages between SX and MF were similar for BMP formulations. BMP fixed dose combinations for co-deposition produced dose proportional aerosol of SX with equivalent performance of the other component MF by percentage deposition. Micronized blends exhibited non-uniformed distribution between SX and MF with the same components consisted as shown in FIGS. 8A-8E. Moreover, the micronized blends were deposited primarily in the capsule and throat, while BMP formulations were deposited primarily in the pre-separator and stage 1. BMP SXMF and BMP SXMFGly exhibited higher FPF and lower MMAD.

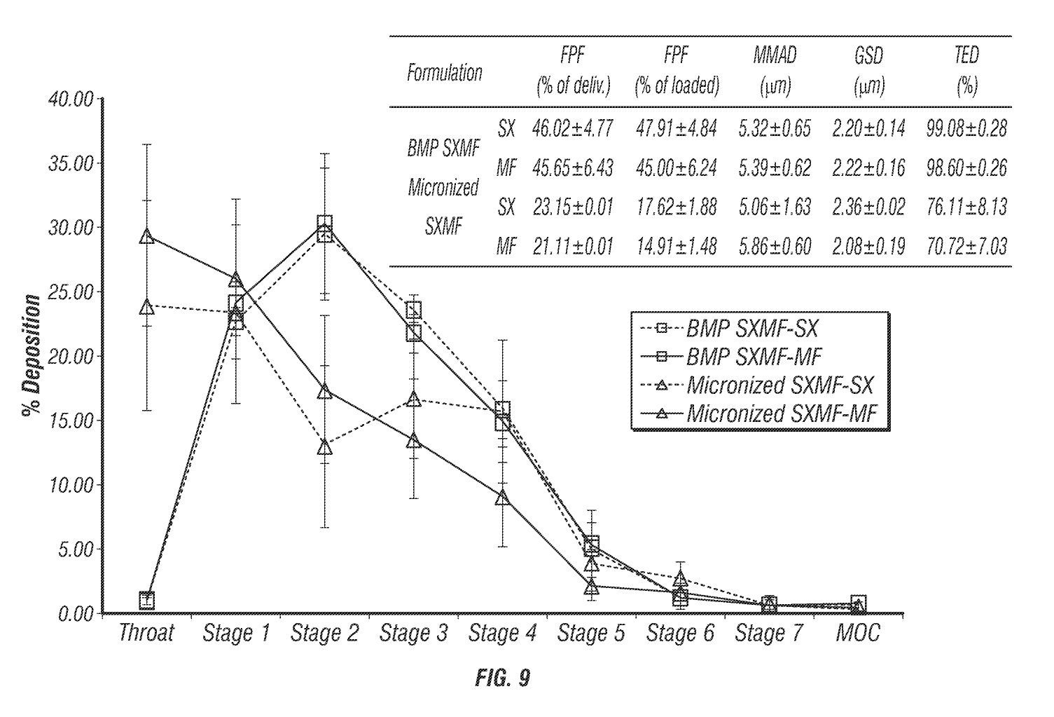

The aerodynamic properties of BMP SXMF and micronized SXMF performed by insufflator using 51 L/min air flow rate through the NGI apparatus were reported in FIG. 9. The FPFs of BMP SXMF were 46.02±4.77% and 45.65±6.43% for SX and MF, and the FPFs of micronized SXMF were 23.15±0.01% and 21.11±0.01%. Though the values of MMAD and GSD were close for both formulations, TEDs of BMP SXMF were almost 99%.

G. Chromatography

Linearity test solutions of SX and MF were conducted with seven concentrations in the range of 1 μg/mL to 250 μg/mL (r>0.99). The recovery rate (%) was between 85%-105%, which was considered reliable, otherwise the test was repeated.

In chiral chromatography, two peaks were observed for all fixed dose combinations and the peak areas were similar indicating that the chirality was not changed after lyophilization, and the retention time of SX chirality was approximately 18 minutes and 21 minutes.

### Example 4—Discussion for SX and MF Combination Therapy

The combo-BMP of SX and MF (in mass ratio of 5:22) was produced by TFF to enable co-deposition of two APIs in consistent ratio throughout the airway. Jet milling was used in order to prepare micronized reference blends for comparison. Lactose (Lac), mannitol (Man), glycine (Gly) and trehalose (Tre) were employed to prepare SX and MF BMP formulations. Unlike coarse blended DPI products which use excipients as carrier particles, the pharmaceutical excipient in BMP form the matrix structure and stabilize amorphous API. Lactose, mannitol and glycine are approved by the U.S. Food and Drug Administration (FDA) as excipients for inhalation and are used in DPIs in differing amounts (e.g., up to 25 mg of lactose (Watts et al., 2013), 0.051% of mannitol and 0.01% of glycine). Trehalose is widely used in pharmaceutical formulations as a lyoprotectant because of its relatively high T_{g} and low tendency to crystallize (Simperler et al., 2006). On the molecular level, SX, MF and the excipients used herein have functional groups that provide hydrogen bond donors and acceptors, such as carboxylic acid, amides, and alcohols, which have been shown to improve the stability of amorphous systems when used in combination (Jeffrey G. A., 1997).

To formulate BMP combinations for co-deposition, it is desirable for SX and MF to be thermodynamically miscible after TFF processing. The appearance of a single T_{g} and recrystallization peak as well as absence of melting peak for two APIs of BMP formulations confirmed the formation of API-API homogeneous amorphous phase, where one API was dissolved in the other API or the excipients. Based on DSC thermograms, it is suggested that BMP SXMF was transferred into co-amorphous state. Thermal properties of excipients were not clear enough to be estimated, because of API degradation. However, the morphological state of individual components is clearer in XRPD studies. XRPD patterns of BMP indicate amorphous API in agreement with DSC thermograms. The crystallography of excipients is also clearly observed. As seen in FIG. 4A, the absence of the characteristic SX and MF crystalline peaks and the appearance of typical halo diffraction patterns in BMP formulations suggested an amorphous morphology of SX and MF. These results indicated amorphous binary (BMP SXMF) or ternary (BMP SXMFLac or BMP SXMFTre) solid dispersions were formed by the introduction of high T_{g} components, that the T_{g} of MF, Lac and Tre was approximately 150° C., 116° C. (Craig et al., 2000) and 94° C. (Simperler et al., 2006). Though SX and MF were amorphous in BMP formulations containing crystalline Gly and Man, crystallization of API may occur over time due to nucleation and crystal seeding (Sun et al., 2012).

Co-amorphous solids were produced to increase the solubility, stability and bioavailability, thus amorphous APIs must resist their thermodynamic tendency to crystallize in order to maintain these advantages. The increased stability of amorphous systems was generally explained by the increased T_{g}, which may reduce the molecular mobility required for crystallization at certain storage temperatures (Janssens and Van den Mooter, 2009). In this study, the BMP SXMF and BMP SXMFTre still remained in good co-amorphous conditions after stored at 25° C./30% RH for 6 months. However, a small SX crystalline peak at 4.21 2θ was observed for BMP SXMFLac, BMP SXMFMan and BMP SXMFGly over the same time period.

Without wishing to be bound by any theory, it is believed that the specific surface area and morphology of API particles can significantly influence the API release characteristics through both kinetic and thermodynamic effects (DiNunzio et al., 2008). The difference in size, density, specific surface area and morphology can be attributed to the process of particle formation (Ali and Lamprecht, 2014). Unlike irregular and cohesive micronized blends, an advantage of BMP formulations was the porous and fragile structure facilitates the potential for rapid dissolution of the API in pulmonary fluid (Janssens and Van den Mooter, 2009). The SSA of BMP formulations were greatly increased compared to the micronized formulations due to the porous matrix structure. All TFF processed powders exhibited very low bulk densities and were expected to aerosolize readily.

Moisture sorption plays a key role in the aerosol characteristics of BMP formulations. It has been reported that respirable BMPs prepared with lactose as excipient by TFF were susceptible to moisture induced matrix collapse and hygroscopicity; in contrast, the aerosolization properties of BMPs were not influenced by high humidity when processed with mannitol (Watts et al., 2013). Without wishing to be bound by any theory, it is believed that hygroscopic excipients may increase the risk of instability in co-amorphous BMP formulations (based on the stability study), while BMP formulations processed with non-hygroscopic excipient or no excipient were more robust (as revealed from in vitro aerosol performance).

Fourier transform infrared spectroscopy (FTIR) measurements were carried out to gain insight into possible molecular level interactions, between SX, MF and excipients in BMP formulations. Crystal single ingredients were analyzed in order to make an easy comparison in this study (Heinz et al., 2009). It was impossible to determine if there was shift because of hydrogen bonding interactions in the region of 3000 cm⁻¹ and 3500 cm⁻¹ corresponding to the —OH stretching, due to the effects of thermal excitation on these vibrational modes for excipients.

Though the strong —NH and —OH peaks in the region of 3200 cm⁻¹ and 3400 cm⁻¹ for the single materials changed shapes, no significant peak shifts could be detected for either —C═O or —NH and —OH in all BMP fixed dose combinations. Thus, wishing to be bound by any theory, it is believed that no interaction occurred. Though hydrogen-bond interactions are usually observed in co-amorphous formulations especially in the formulations composed of materials structured with potential hydrogen receptors and donors, it was not inevitable for this to occur and not necessary for improving their physiochemical properties (Dengale et al., 2014; Lobmann et al., 2012).

Aerodynamic performance is one factor in measuring the performance for inhaled dry powder formulations as it is correlated to lung deposition (Ali and Lamprecht, 2014). Though the BMP formulations produced large geometric diameters, low particle density allows for sufficiently low aerodynamic diameters for effective lung deposition (Carvalho et al., 2014; Watts et al., 2013). The Miat monodose Inhaler® was employed herein, which provided the necessary shear force to break up the brittle matrices into respirable particles. In addition, powders delivered from a powder insufflator were characterized in the preparation for the in vivo study.

More BMP formulation emptied from the capsule and adaptor than the micronized blends, as demonstrated by the higher TED. The FPF (% of delivered) of SX in BMP formulations was improved by 25% compared to a previously investigated SX combination formulation prepared by anti-solvent precipitation (Mumane et al., 2009). For the monotherapy MF Twisthaler, it has been reported that the FPF of 200 μg strength is 27.9% (Berger and Berger, 2013), thus the FPF of MF-DPI was increased significantly as well in this study. The data presented in Table 4 showed that the aerosol properties of SX and MF for BMP formulations were similar, suggesting that both APIs were co-deposited after inhaled in vitro. Conversely, micronized SX and MF delivered in a combination powder blend demonstrated different FPFs and MMADs.

Aerodynamic particle size distribution within the NGI, shown in FIG. 8A-8E suggested that APIs of BMP formulations were delivered to all stages with highly consistent, while micronized blends resulted in variance. It has been reported that after administration of SX and a corticosteroid fluticasone propionate from a single inhaler, co-location on the cellular level offers a potential for increased clinical efficacy (Nelson et al., 2003). Thus, it may be that this fixed dose combination offers increased opportunity for synergistic interaction to occur than the same dose of two separate inhalers.

Powders formulated with non-hydroscopic excipients or no excipient showed elevated FPFs compared to hydroscopic powders, for example BMP SXMF versus BMP SXMFLac as shown in Table 4. Based on the comparison of deposited percentage for SX and MF, without wishing to be bound by any theory, it is believed that reduced surface cohesion of BMP formulations lead to low capsule retention which was normally caused by van der Waals, capillary, and electrostatic forces in traditional formulations (Watts et al., 2013). Though FPFs as a percentage of loaded dose were around 38%-57% for all two TFF formulated APIs, further optimization of formulation/device is needed to reduce upper airway (stage 1 and 2) deposition in these formulations. BMP SXMF powder was chosen for in vivo study due to its stability and good aerodynamic performance, meanwhile crystalline micronized SXMF blend was employed as reference. Moreover, the in vitro aerosol performance study suggested that insufflator could produce fine aerosol particles for animal study with both formulations.

The fixed dose combination BMP SXMF exhibited increased deposited amount of both APIs in lung tissue and increased bioavailability significantly in lung and blood. Further formulation approaches could be considered to decrease the extent of MF systemic absorption. Since long-acting β2-agonists (LABA) have the ability to prime glucocorticosteroid receptors and improve the activity of inhaled corticosteroids, the fixed dose combination BMP SXMF had potential better pharmacodynamics than traditional crystalline micronized SXMF blends.

### Example 5—Methods and Materials for Triple Therapy

A. Materials

Budesonide, formoterol fumarate and tiotropium bromide were purchased from Chemieliva Pharmaceutical Co. (Chongqing, China). D-(+)-Mannitol was purchased from Acros Organics (Geel, Belgium) and lactose monohydrate (Lactohale® LH 200) was kindly donated by Friesland Foods Domo (Zwolle, Netherlands). High performance liquid chromatography (HPLC) grade acetonitrile was purchased from Fisher Scientific (Fair Lawn, N.J.) and perchloric acid 8% w/v aqueous was purchased from Ricca Chemicals (Arlington, Tex.). Water was purified by reverse osmosis (MilliQ, Millipore, France).

B. Formulation Preparation

Thin Film Freezing technology was used for the preparation of low-density dry powder as described herein and in Overhoff et al. (2007). Triple combo formulations were prepared using the weight ratio of 1:2:35.5 for formoterol, tiotropium and budesonide, respectively. The ratio chosen was based on the typical doses used to treat COPD patients, according to the Global initiative for chronic Obstructive Lung Disease (GOLD), Inc. The following mixtures were prepared by dissolving the components in a co-solvent mixture of three parts of acetonitrile and two parts of water: budesonide/tiotropium/formoterol and mannitol (BTF_Man), budesonide and mannitol (Bud_Man), tiotropium and mannitol (Tio_Man), formoterol and mannitol (For_Man), budesonide/tiotropium/formoterol and lactose (BTF_Lac), budesonide and lactose (Bud_Lac), tiotropium and lactose (Tio_Lac), formoterol and lactose (For_Lac). The ratio of drug(s) to sugar excipient was 1 to 1 and the final solid loading concentration was 0.50% (w/v).

Solutions were rapidly frozen on a cryogenically cooled (−80° C.) stainless steel surface using the thin film freezing apparatus. The frozen disks were collected in a container filled with liquid nitrogen to avoid melting. The frozen formulations were transferred to a −70° C. freezer until complete evaporation of the liquid nitrogen and then transferred to a VirTis Advantage Lyophilizer (VirTis Company Inc., Gardiner, N.Y.) for solvent removal. Formulations were lyophilized over 24 h at −40° C. at pressure of 400 mTorr, temperature was gradually ramped to 25° C. over 24 h with pressure less than 200 mTorr, and kept at 25° C. for 24 h.

For comparison purposes, the equivalent physical mixtures of budesonide/tiotropium bromide/formoterol fumarate and mannitol (BTF_Man_PM) and budesonide/tiotropium bromide/formoterol fumarate and lactose (BTF_Lac_PM) were prepared using the same weight ratio as described previously. The actives were micronized using a fluid energy laboratory jet-o-miser (Fluid Energy, Telford, Pa.) with pusher pressure set at 80 psi and the gridding pressures set at 100 psi. Precise amounts of micronized powders were weighed and mixed using the geometric dilution technique and then transferred to a stainless steel vessel. The vessel was placed in a Turbula mixer (Glen greston Ltd., Middx, UK) and mixing was carried out for 20 min at 45 revolutions per minute (rpm). Formulations were sieved through 100 and 45 μm mesh size before and after mixing.

C. Thermal Analysis

The TA Instruments modulated Differential Scanning Calorimeter (mDSC 2920) (New Castle, Del.), equipped with a refrigerated cooling system, was used to analyse the thermal properties and the degree of crystallinity of the powders. Dry nitrogen was used as the purge gas for the mDSC cell at a flow rate of 40 mL/min. Sample weights of 4 to 10 mg were placed into open aluminum pans and hermetically sealed (kit 0219-0041, Perkin-Elmer Instruments, Norwalk, Conn.). Experiments were carried out in the range of 10 to 350° C. at a heating rate of 10° C./min and modulation temperature amplitude of 1° C./min. Data was analyzed using TA Universal Analysis 2000 software (TA Instruments, New Castle, Del.).

D. Powder X-Ray Diffraction (PXRD)

Crystallinity properties of the dried powders of raw actives as purchased as well as the jet milled and TFF powder formulations were investigated using a Philips 1710 X-ray diffractometer (Philips Electronic Instruments Inc. Mahwah, N.J.). Measurements were taken from 50 to 350 on the 2-theta scale at a step size of 0.03°/s and a dwell time of 5 s.

E. Particle Size Analyses

Measurement of particle size distributions of budesonide, tiotropium bromide and formoterol fumarate before and after jet milling were measured by laser diffraction (HELOS, Sympatec GmbH, Clausthal-Zellerfelg, Germany). A small amount of formoterol fumarate and Tiotropium bromide were separately dispersed in 10 mL 0.01% tween 80 mineral oil and a small amount of budesonide was dispersed in 10 mL 0.01% tween 80 in deionized water. The samples were sonicated for 5 minutes and diluted with enough solvent to produce light obscuration in the range of 15-20%. The sizes reported are average values of at least 3 measurements. The results are presented as D₍ₓ₎ and span, where X is the cumulative percentile of particles under the referred size (e.g. D₍₅₀₎ corresponds to the median diameter of the particles). Span is a measurement of particle size distribution calculated as [(D₍₉₀₎−D₍₁₀₎]/D₍₅₀₎.

F. Scanning Electron Microscopy (SEM)

Powder morphologies and estimation of particle sizes were determined using a SEM. Samples were placed on carbon tape and coated with gold/palladium (60/40) for 20 seconds under high vacuum using a Cressington 208 Benchtop Sputter Coater (Watford, England). The SEM images were captured using a SmartSEM® graphical user interface software in a Carl Zeiss Supra® 40VP (Carl Zeiss, Oberkochen, Germany) operated under vacuum, at a working distance of 19 mm and at 5 kV of Electron High Tension (EHT).

G. Brunauer-Emmett-Teller (BET) Specific Surface Area (SSA) Analysis

Powder porosity was determined through the measurement of the specific surface area (SSA) using a Monosorb MS-22 rapid surface area analyzer (Quantachrome Instruments, Boynton Beach, Fla.). The instrument uses a modified BET equation for SSA determination. Samples were degassed in a Thermoflow™ Degasser for at least 2 hours at 25° C. using 30% nitrogen in helium as the desorbate gas.

H. Fourier Transform Infrared Spectroscopy (FTIR)

FTIR spectroscopy was used to characterize chemical interactions and/or amorphous and crystalline polymorphs of each sample. FTIR scans of dry samples were collected on a Nicolet IR100 spectrometer (Thermo Fisher Scientific, Pittsburgh, Pa.) equipped with a Deuteriated tri-glyceride sulfate (dTGS) detector. KBr disc method was used with approximately 1% (w/w) sample loading. A total of 32 scans were accumulated at a resolution of 4 cm⁻¹ in the region of 4000 to 600 cm⁻¹.

I. In Vitro Aerosol Performance

Aerodynamic particle size distribution and deposition homogeneity were evaluated by the Next Generation Cascade Impactor (NGI) (MSP Corporation, Shoreview, Minn.) using a Handihaler® device attached to the induction port by a mouthpiece adaptor made of silicon. The cascade impactor was assembled and operated in accordance to the USP General Chapter <601> Aerosol, Nasal Spray, Metered-dose Inhalers and Dry Powder Inhalers. The device was run for 4.4 seconds at a pressure drop of 4 kPa across the device corresponding to a flow rate of 54 L/min, which was calibrated using a TSI mass flowmeter (Model 4000, TSI Inc., St. Paul, Minn.). The NGI collection plates were coated with 1% (v/v) silicone oil in hexane to prevent particle bounce, fracture and reentrainment. Three capsules were fired in sequence into the NGI and the experiments were performed in triplicate for each formulation under investigation. After aerosolization, samples were collected using known volumes of diluent and analyzed by high performance liquid chromatography (HPLC).

Emitted dose (ED) was calculated as the percentage of drug emitted from the DPI. Fine particle fraction (FPF) was calculated as the sum of assayed dose deposited on stages 2 through micro-orifice collector (MOC) corresponding to particles with and aerodynamic diameter≤4.46 μm. Mass median aerodynamic diameter (MMAD) was calculated via regression of a log-probability plot of cumulative percent versus cut-off diameter and geometric standard deviation (GSD) was calculated as the square root of the 84^{th}/16^{th} percentile.

J. HPLC Assay

Chemical analyses of all drugs were performed using a Dionex 3000 HPLC system equipped with UV detector set at 230 nm wavelength. A 20 μL injection volume was injected into an Inertisil C₈ 5 μm 150×4.6 mm reversed-phase column (Thermo Fisher Scientific, Waltham, Mass.) maintained at 26° C. Gradient elution was used and the mobile phase consisted of a 0.2% v/v perchloric acid solution as solvent-A and acetonitrile as solvent-B, running at a flow rate of 1.2 mL/min and run time of 10 minutes, as described elsewhere (17). The method was tested with regard to variability, recovery, linearity, detection limit and range, and shown to be suitable for this study.

K. Statistical Analysis

The data is expressed as a mean±standard deviation (SD). Statistical analyses were performed using NCSS/PASS software Dawson edition. Significant differences between formulations and between the percentage distributions of all three drugs on the NGI stages were analyzed using One-way ANOVA (p<0.05).

### Example 6—Results and Discussion for Triple Therapy

A. Particle Size and Morphology of Formulations

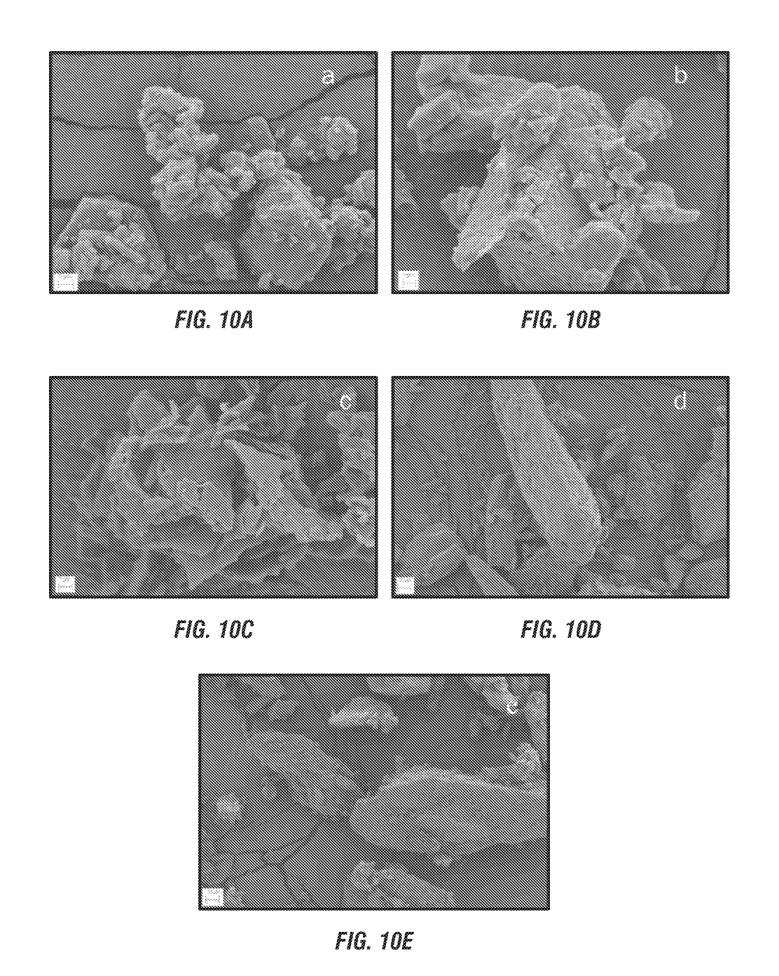

Particle size analyzes by laser diffraction and scanning electron microscopy images of the bulk drug and excipient powders showed that the sizes of the particles are not suitable for lung delivery. SEM images of budesonide and tiotropium display irregular shape with D₅₀ values of 22.11±16.92 and 5.14±0.03 μm respectively and broad particle size distributions confirmed by the large span values of 2.31 and 2.14 (Table 5 and FIGS. 10A & 10B). Formoterol, on the other hand, displays a plate-like shape with D₅₀ values of 6.27±0.97 μm and greater span value of 7.75 (Table 5 and FIG. 10C). In addition, specific surface area measurements (SSA) gave small surface area values of 3.05±0.21 m²/g for budesonide, 2.08±0.13 m²/g for tiotropium and 2.97±0.05 m²/g for formoterol indicating that the bulk powders contain large particles.

TABLE 5
Particle size distribution and specific surface area of bulk drugs and
excipients, jet milled drugs and TFF formulations.

| | Particle size (μm) | | | | SSA ± SD |
|---|---|---|---|---|---|
| Samples | D₁₀ | D₅₀ | D₉₀ | Span | (m²/g) |
| Budesonide | 1.63 ± 1.25 | 22.11 ± 16.92 | 52.7 ± 2.42 | 2.31 | 3.05 ± 0.21 |
| Tiotropium bromide | 1.10 ± 0.01 | 5.14 ± 0.03 | 12.11 ± 0.07 | 2.14 | 2.08 ± 0.13 |
| Formoterol furmarate | 1.16 ± 0.05 | 6.27 ± 0.97 | 49.78 ± 7.84 | 7.75 | 2.97 ± 0.05 |
| Lactose monohydrate | 4.13 ± 0.11 | 39.78 ± 1.32 | 100.9 ± 0.27 | 2.43 | 0.34 ± 0.03 |
| Mannitol | 6.88 ± 0.07 | 52.70 ± 0.59 | 101.91 ± 0.41 | 1.8 | 0.22 ± 0.001 |
| Jet milled budesonide | 0.91 ± 0.01 | 3.40 ± 0.09 | 9.7 ± 0.30 | 2.58 | 2.75 ± 0.13 |
| Jet milled tiotropium bromide | 1.09 ± 0.79 | 4.65 ± 0.04 | 10.39 ± 0.04 | 2.0 | 2.86 ± 0.11 |
| jet milled formoterol fumarate | 1.45 ± 1.01 | 4.51 ± 0.02 | 10.29 ± 0.07 | 1.96 | 5.08 ± 0.26 |
| BTF_Lac_PM | — | — | — | | 0.43 ± 0.05 |
| BTF_Man_PM | — | — | — | | 0.32 ± 0.03 |
| Bud_Man | — | — | — | | 46.97 ± 2.22 |
| Tio_Man | — | — | — | | 38.79 ± 0.81 |
| For_Man | — | — | — | | 64.14 ± 0.68 |
| BTF_Man | — | — | — | | 54.55 ± 1.10 |
| Bud_Lac | — | — | — | | 90.39 ± 6.15 |
| Tio_Lac | — | — | — | | 48.90 ± 2.42 |
| For_Lac | — | — | — | | 193.86 ± 10.63 |
| BTF_Lac | — | — | — | | 81.31 ± 1.71 |

Coarse lactose and mannitol were used as carriers to enhance powder dispersion at time of aerosolization. SEM images of both powders show irregular shape with D₅₀ values of 39.78±1.32 μm for lactose and 52.70±0.59 μm for mannitol. Although the particle sizes of lactose and mannitol were bigger than those of the raw drugs, nevertheless, the particle size distributions were similar with span values of 2.43 and 1.80, respectively (Table 5 and FIGS. 10D & 10E).

In order to prepare the triple combo physical mixture formulations for comparison purposes with the TFF formulations, particle size reduction of the three drug powders was necessary. Laser diffraction results indicate that the size reduction process was only significant for bulk budesonide powder with D₅₀ values of 3.40±0.09 μm and broad particle size distribution with span value of 2.58. The SSA of bulk budesonide decreased to 2.75±0.13 m²/g regardless of the reduction in particle size (Table 5). Without wishing to be bound by any theory, it is believed that this may be due to aggregation of the micron size particles, which can be caused by cohesive forces generated during the uncontrolled milling process resulting in electrostatically charged particles with heterogeneous shapes as shown FIG. 11A. Laser diffraction results of tiotropium and formoterol show a slight reduction in particle size after milling process with D₅₀ values of 4.65±0.04 and 4.51±0.02 μm, respectively. Moreover, particle size distribution of tiotropium shows a small reduction with span value of 2.0 where as formoterol showed a significant reduction in particle size range with span value of 1.96 (Table 5). SEM images of tiotropium and formoterol show agglomerated micronized powders with irregular shapes and the SSA results confirm the reduction in particle sizes with increased values to 2.86±0.11 and 5.08±0.26 m²/g, respectively (FIGS. 11B & 11C). SEM images of the physical mixture of micronized drugs with coarse lactose and coarse mannitol are shown in FIGS. 11D and 11E. The images show micronized drug powders adhered to the coarse lactose and mannitol surfaces, which may improve powder dispersion and aerosolization at the time of inhalation. Due to the mixture with coarse lactose and mannitol, the physical mixture formulations produced small SSA values of 0.43±0.05 and 0.32±0.03 m²/g (Table 5).

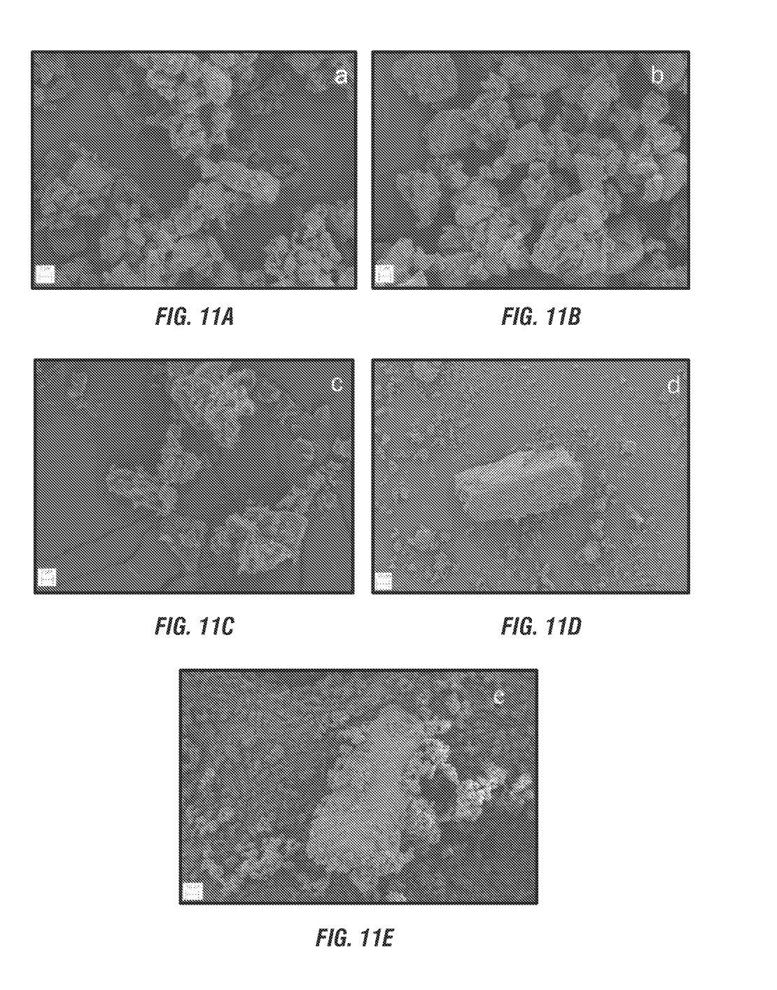

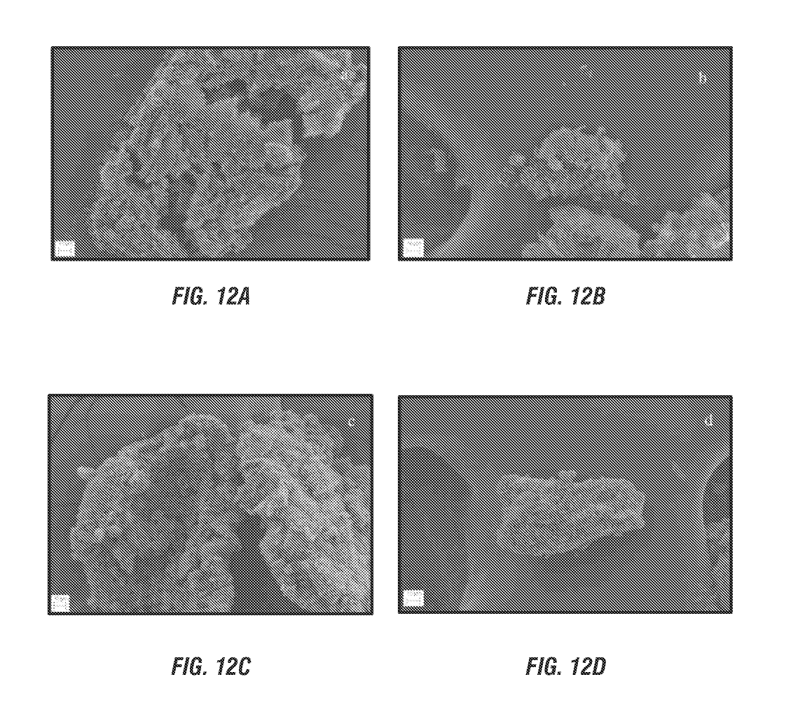

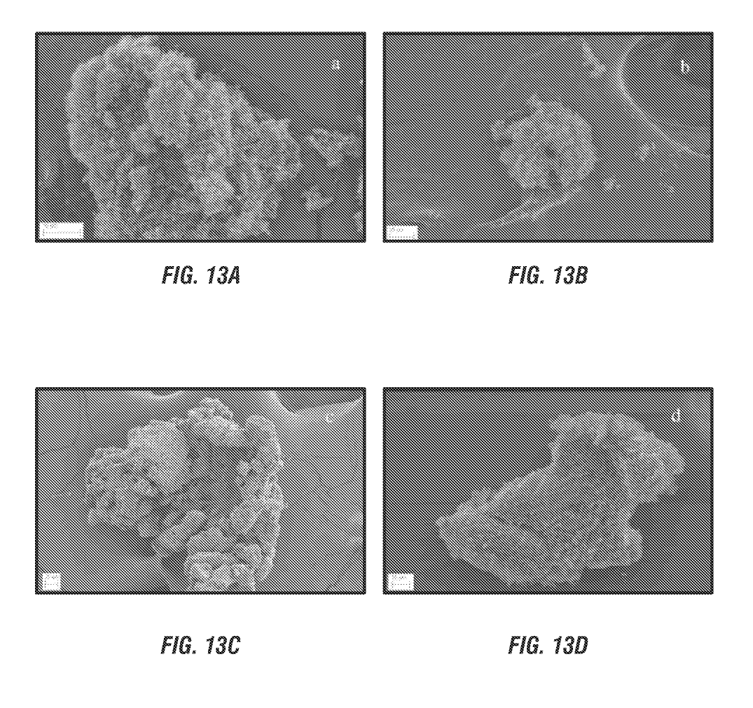

SSA measurements of TFF powder formulations show a significant increase in surface area due to the highly porous cake powder formed after lyophilization of the frozen discs (Table 5). Tiotropium formulations produced the least porous cakes with SSA values of 38.79±0.81 m²/g for Tio_Man and 48.90±2.42 m²/g for Tio_Lac. The SSA values for budesonide were much higher than those of the tiotropium formulations with values of 46.97±2.22 m²/g for Bud_Man and 90.39±6.15 m²/g for Bud_Lac. Formoterol formulations, however, presented the greatest SSA values of 64.14±0.68 m²/g for For_Man and 193.86±10.63 m²/g for For_Lac. The triple therapy formulations also produced large SSA values of 54.55±1.10 m²/g for BTF_Man and 81.31±1.71 m²/g for BTF_Lac. These values are very similar to the SSA values of budesonide formulations, as the triple therapy formulation has a high percentage of budesonide in the formulation (Table 5). The difference in powder density and porosity can be seen in the SEM images. Powder formulations with greater SSA values also show a more porous cake structure as shown in FIGS. 12A-12D & 13A-13D.

B. Crystallinity Evaluation

1. Budesonide

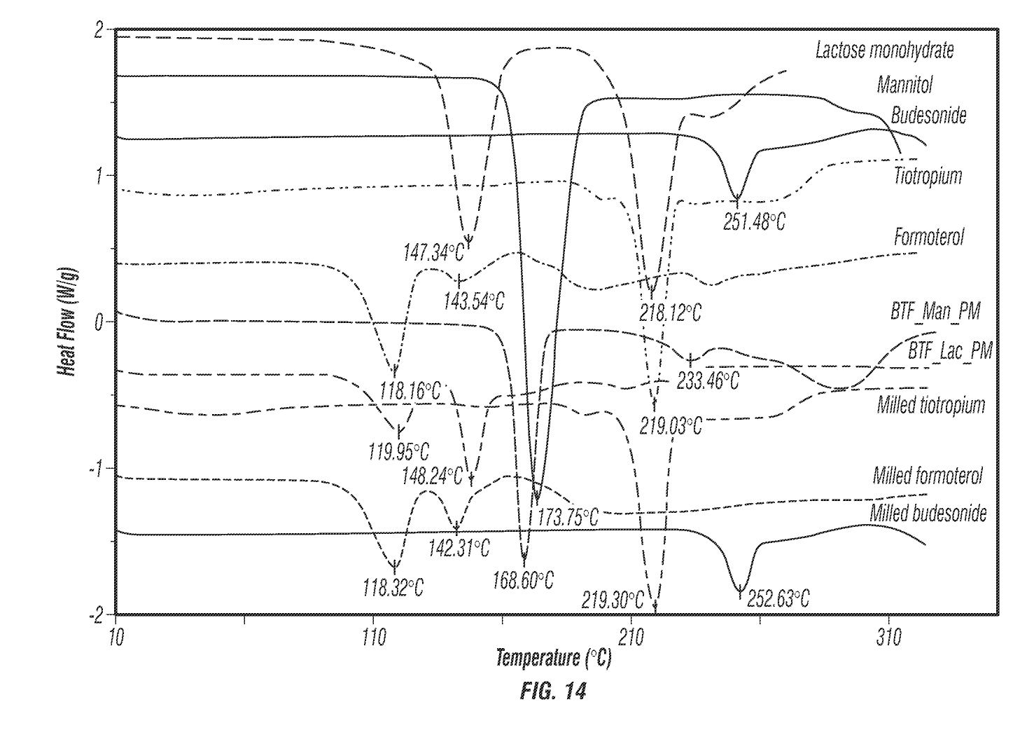

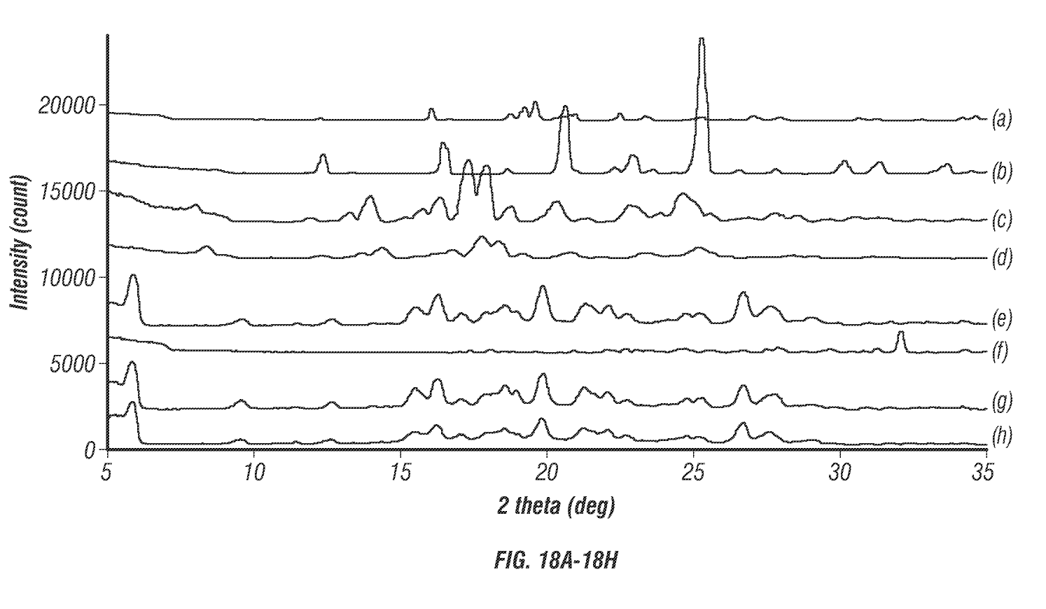

Budesonide was supplied as a micronized powder. PXRD pattern of budesonide exhibit high intensity peaks at 11.810, 15.2°, 15.77°, 18.14° and 22.46° of 2θ, indicating its crystalline structure, which is in accordance with data reported by Tajber et al., as shown in FIG. 18C (Tajber et al., 2009). The mDSC profile of bulk budesonide powder shows a single endothermic melting peak at 251.4° C. (FIG. 14) similar to the data reported by Velaga et al. confirming its crystalline properties (Velaga et al., 2002). After jet milling process, micronized budesonide powder remained crystalline as shown by mDSC peak profile (FIG. 14) and PXRD pattern (FIG. 18C). The mDSC profile shows a slight shift of the endothermic melting peak with peak maxima at 252.6° C. Accordingly, PXRD pattern shows a reduction in the peak intensities but the sample remains mostly crystalline. The observed shift of endotherm peak and the reduction of the PXRD peak intensities may be a result of a change in the crystalline structure of the powder at the time of comminution. The milling process disrupts the crystal structure on the particle surface and creates amorphous domains (Rasenack and Muller, 2004).

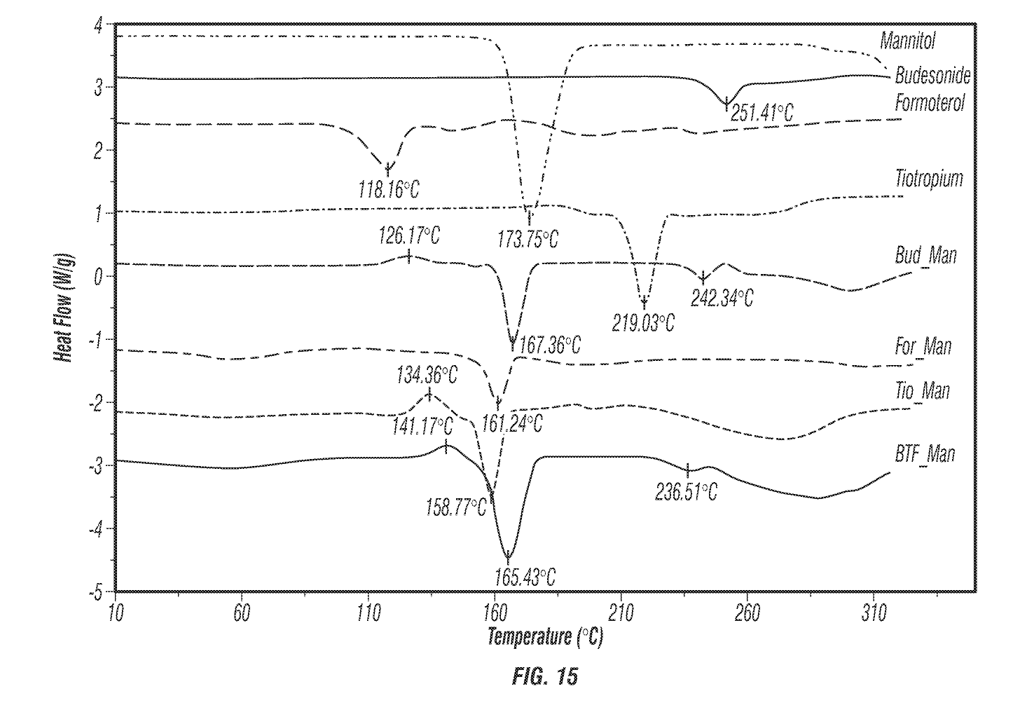

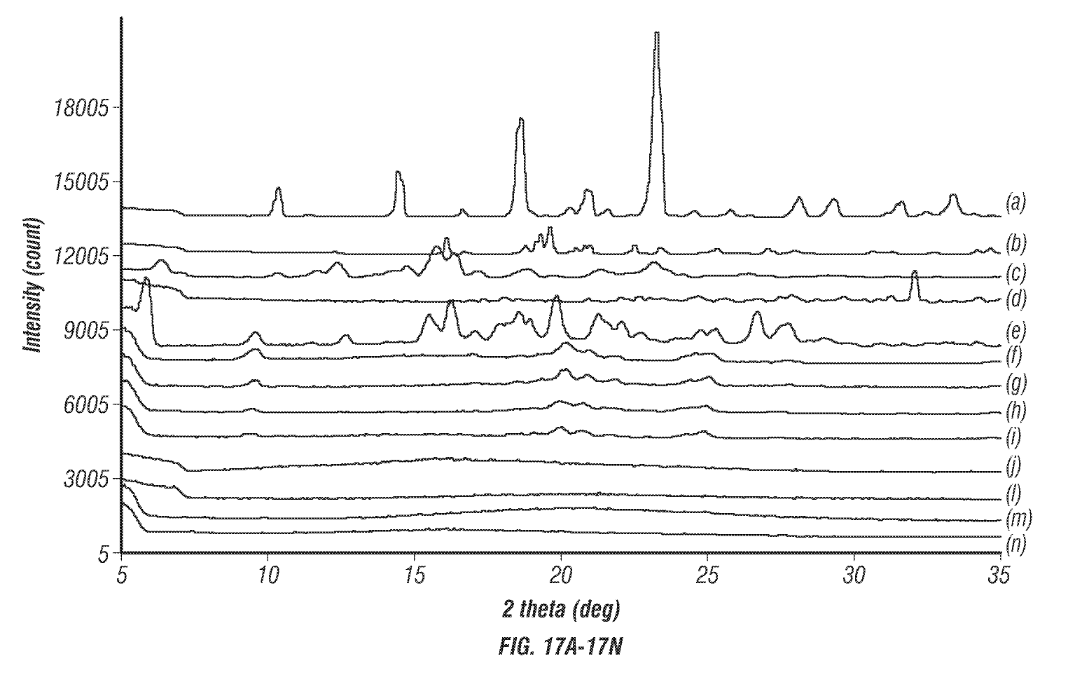

Thin film freezing (TFF) of budesonide formulation prepared with mannitol yielded partially crystalline powders, as confirmed by the mDSC (FIG. 15) and PXRD (FIG. 17F) results. The PXRD pattern of Bud_Man shows peaks with small intensities characterizing a partially crystalline formulation. From the mDSC profile, Bud_Man exhibit one exothermic recrystallization peak at 126.2° C. and two endothermic melting peaks, one at 167.4° C., corresponding to the melting point of mannitol and the second at 242.3° C. corresponding to the melting of budesonide. These results suggest a partially amorphous nature of the Bud_Man formulation. Kim et al. investigated the physicochemical characteristics of mannitol after lyophilization and reported that lyophilized mannitol yields a partially crystalline powder as a consequence of the low glass transition temperature of the pure amorphous powder, which is observed at 13° C. This disclosure also suggests that the relative concentration of crystalline mannitol in the formulation should be above 30% (w/w) in order to be detected by PXRD (Watts et al., 2013).

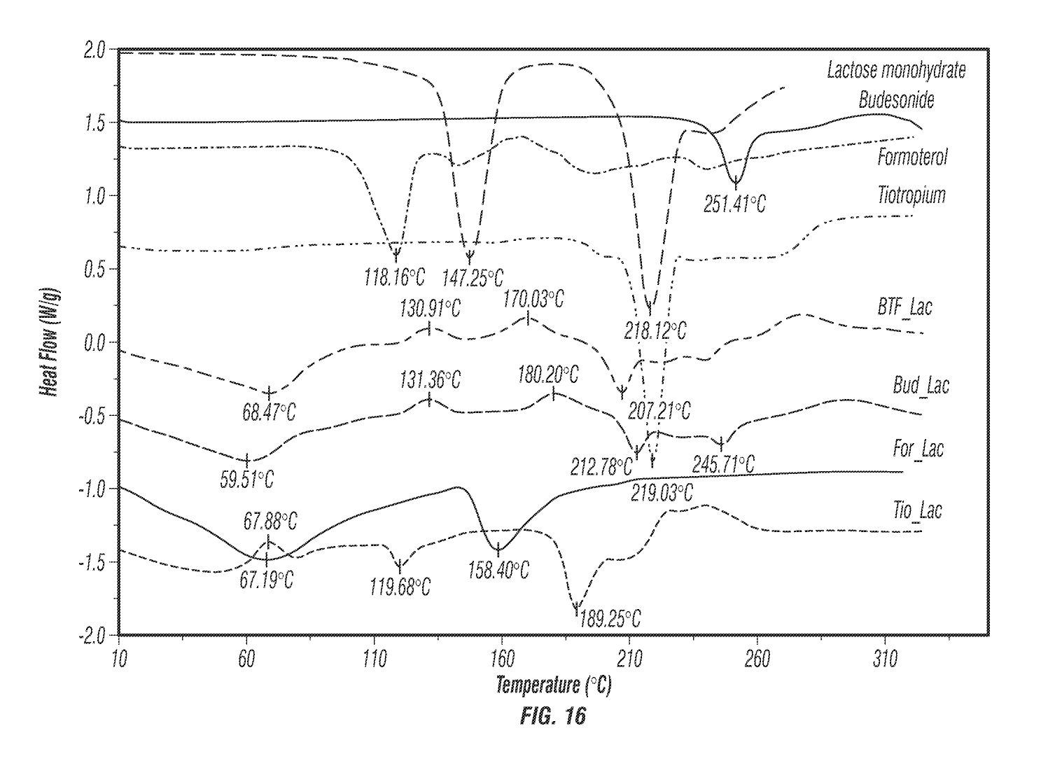

Regarding the PXRD pattern of TFF Bud_Lac formulation, shown in FIG. 17J, broad and diffuse halos were present with an absence of the characteristic crystalline peaks, indicating an amorphous structure. FIG. 16 shows the modulated DSC thermogram which indicates two recrystallization events, in which the first peak may represent the recrystallization of lactose at 131.4° C., and the second peak may represent the recrystallization of budesonide at 180.2° C. The recrystallization events confirm that an amorphous structure is formed by the ultra rapid freezing process. Two endothermic melting peaks are also observed, which may correspond to the melting of lactose at 212.8° C. and budesonide at 245.7° C. The two exothermic and endothermic peaks may indicate the formation of a solid dispersion system and a posterior phase separation of budesonide and lactose at the time of analysis (Overhoff et al., 2007).

2. Tiotropium Bromide

The PXRD pattern of tiotropium bromide exhibits crystalline high intensity peaks at 5.810, 16.13°, 19.79°, and 26.6° of 2θ (FIG. 18E). The crystallinity of this sample is confirmed by the mDSC profile of the bulk tiotropium powder, which exhibits a single endothermic melting peak at 219.0° C. (FIG. 14). Micronized tiotropium powder obtained by jet milling remained crystalline, indicated by the absence of an exothermic recrystallization peak and the presence of an endothermic melting peak at 219.3° C. in the mDSC profile (FIG. 14). However, the PXRD pattern shows a significant decrease in the intensity of crystalline peak diffractions, which may be as a result of the loss of powder crystallinity and the formation of amorphous domains (Alhalaweh et al., 2012).

The TFF Tio_Man formulation, which was prepared with mannitol as a stabilizing sugar, has also shown to be partially crystalline, as observed on the PXRD by less intense crystalline peak diffractions which are similar to those of tiotropium and mannitol (FIG. 17G). The partial crystallinity of Tio_Man is confirmed by an exothermic recrystallization peak at 134.3° C. followed by an endothermic melting peak at 158.8° C. (FIG. 15). Only a single recrystallization and melting peak were present. The PXRD pattern of the TFF Tio_Lac formulation is shown in FIG. 17L. Similar to Bud_Lac, the Tio_Lac powder exhibits broad and diffuse halos with an absence of the characteristic crystalline peaks indicating an amorphous structure. The amorphous form of the powder is confirmed by the mDSC thermogram (FIG. 16). The thermogram profile shows an exothermic recrystallization event at 67.9° C., and two endothermic melting peaks at 119.7° C. most likely due to lactose and at 189.2° C. most likely a result of tiotropium.

3. Formoterol Fumarate

The PXRD pattern of formoterol fumarate is shown in FIG. 18G where crystalline high intensity peaks can be seen at 5.75°, 15.29°, 16.10°, 18.38°, 19.76° and 26.60° of 2θ. The PXRD pattern indicates that the material analyzed is a dihydrate polymorph of formoterol fumarate, as reported by Tajber et al. and Jarring et al. (Vehring et al., 2012; Trivedi et al., 2012). A modulated DSC heat flow thermogram of formoterol shows two endothermic melting peaks occurring at 118.2° C. and at 143.54° C., as shown in FIG. 14. When analyzed by mDSC reverse heat flow, the thermogram of formoterol fumarate displays three endotherm peaks at 111.4, 123.1 and 139.1° C. Tajber et al. have also investigated the thermodynamic properties of formoterol fumarate and reported the findings of three melting peaks. The first and largest peak occurred at approximately 122° C. which was anticipated as a dehydration event. The last two peaks appeared at approximately 130° C. and 150° C. The PXRD pattern of jet milled formoterol shows that the material has remained mostly in the crystalline form with peak diffractions presenting only a slight reduction in intensity as seen in FIG. 18H The mDSC profile confirms the crystalline state of the micronized material showing the first peak at 118.3° C. and the second peak at 142.3° C. (FIG. 14).

The TFF For_Man powder, when subjected to PXRD analysis, displayed small peak diffractions indicating the presence of crystalline structures (FIG. 17H). The crystallinity of the material is also confirmed by the presence of a single endothermic melting peak at 161.2° C., which may be related to the melting of mannitol. The melting peak of formoterol was not observed which suggests that the material was in the amorphous state (FIG. 15). The mDSC reverse heat thermogram analysis of TFF For_Lac powder exhibits a recrystallization peak at 152.9° C. and a following melting peak at 163.8° C. The mDSC heat flow thermogram shows a melting peak at 158.4° C. as well as a peak at 67.19° C. that could represent the glass transition temperature of the formulation (FIG. 16). PXRD confirms the amorphous characteristics of the powder exhibiting a halo pattern with absence of crystalline peaks (FIG. 17M).

4. Triple Drug Combinations

Much like the TFF single drug formulations, the PXRD pattern of TFF BTF_Man has shown to be crystalline exhibiting small intensity diffraction peaks (FIG. 17I). The mDSC thermogram of BTF_Man powder exhibits an exothermic recrystallization peak at 141.2° C. followed by two endothermic peaks. The first and largest peak occurred at 165.4° C., most likely corresponding to the melting of mannitol and the second peak occurred at 236.5° C. possibly representing the melting of budesonide (FIG. 15). Furthermore, the TFF BTF_Lac powder displayed broad and diffuse haloes with an absence of the characteristic crystalline peaks, indicating an amorphous structure. The mDSC thermogram profile is characteristic of an amorphous formulation showing two exothermic recrystallization peaks at 130.9° C. and 170.0° C. This was followed by two endothermic melting peaks at 207.2° C., which may be related to the melting of lactose, and at 245.7° C., which corresponds to the melting of budesonide. Without wishing to be bound by any theory, it is believed that the lactose and budesonide peaks would be more evident in the characterization of the triple combo formulations due to the presence of the largest amount of these materials in the formulation.

C. Analysis of the Samples by FTIR

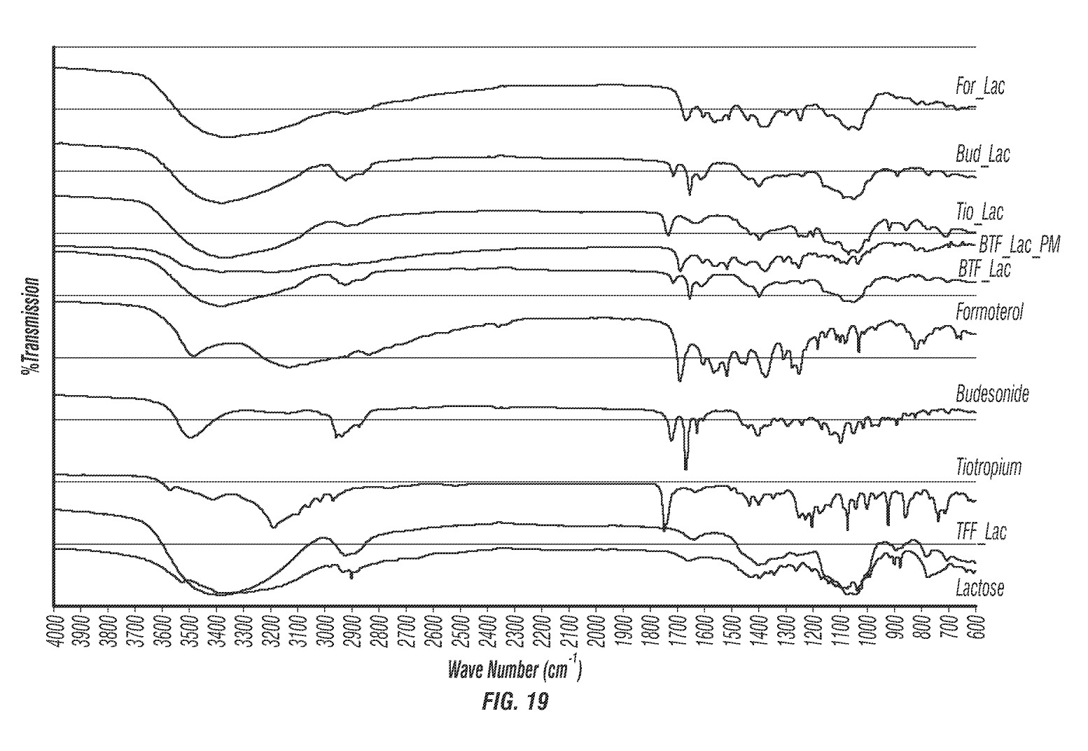

The IR frequencies of OH stretching vibrations are affected by hydrogen bonding of these groups. However, the OH stretching region of the binary- and tertiary mixtures of drugs is dominated by the broad envelopes from the effects of thermal excitation on these vibrational modes for lactose and mannitol. As such, it is difficult to determine if there are shifts due to hydrogen bonding interactions in this region of the spectra. In contrast, the carbonyl-stretching region of the IR spectra provides some insight in to possible hydrogen bonding interactions of these functional groups of the drugs. In the case of tiotropium bromide, there is a significant 20 cm⁻¹ shift in the frequency of the ester carbonyl stretch in the binary Tio_Lac formulation from 1749 to 1729 cm⁻¹. A smaller shift of carbonyl of about 15 cm⁻¹ is observed in the binary Tio_Man formulation from 1749 to 1734 cm⁻¹. These shifts are commensurate with hydrogen bonding interactions, which result in decreases in carbonyl stretching frequency, as shown in FIG. 19. In addition to the apparently stronger hydrogen-bonding interaction between tiotropium and lactose as evidenced by this carbonyl stretching frequency shift, further evidence for interaction comes from analysis of the lower frequency region of the IR spectra. The most pronounced feature in this region is the apparent absence of the lactose ˜900-870 cm⁻¹ bands in the Tio_Lac formulation. These bands are resolved in raw lactose, but appear as a single broad band in TFF-processes lactose. In the binary mixture, these bands may possibly be shifted to ˜860 cm⁻¹, although a 860 cm⁻¹ band also appears in the spectrum for tiotropium alone, it is much less intense that the band from the binary formulation. Because this region of the IR spectrum consists of transitions with substantial coupling, it is difficult to make a conclusive assignment for the origin of this band, and thus to the nature of the interaction that gives rise to its spectral change. However, in light of the evidence for tiotropium carbonyl hydrogen bonding, without wishing to be bound by any theory, it is believed that this change is due to hydrogen bonding interactions with lactose (FIG. 19).

The IR spectra of the binary For_Lac and Bud_Lac formulations show more modest shifts in the carbonyl region. In the case of formoterol, the carbonyl-stretching band at 1687 cm⁻¹ shifts slightly to 1660 cm⁻¹, accompanied by a marked decrease in intensity. The saturated ketone carbonyl-stretching band of budesonide moves slightly from 1722 cm⁻¹ to 1712 cm⁻¹ in the binary formulation. Similarly, the dienone carbonyl also shifts slightly, from 1666 cm⁻¹ to 1650 cm⁻¹, in the binary formulation. These shifts suggest that there is minimal hydrogen bonding to these carbonyl groups in the binary formulation.

The IR spectrum of the BTF_Lac formulation is dominated by the major component budesonide and shows little change in carbonyl stretch peak positions relative to the corresponding binary drug-lactose formulation.

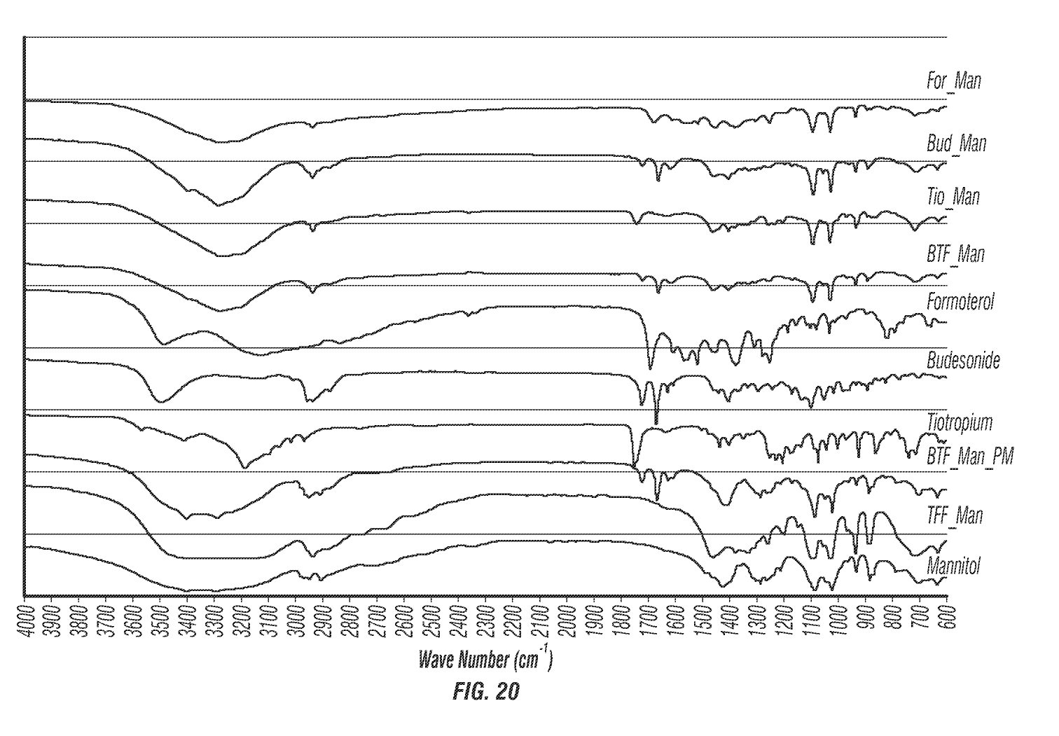

In the case of the formulations with mannitol, a slightly different picture emerges from analysis of the IR spectra. In binary drug-mannitol mixtures, there are slight changes in the carbonyl stretching bands when compared to spectra of the drugs alone, as seen in FIG. 20. For example, the tiotropium carbonyl band shifts from 1749 to 1734 cm⁻¹, the formoterol formamide carbonyl band shifts from 1687 to 1664 cm⁻¹, but the budesonide saturated ketone carbonyl band remains unchanged at 1723 cm⁻¹. In contrast, in the ternary formulation, the saturated ketone carbonyl band for budesonide is even further shifted to lower frequency than in the case of the binary mixture, from 1717 to 1710 cm⁻¹. In contrast, the dienone carbonyl band for budesonide remains unchanged from the binary mixture. Together, these IR band shifts suggest that there is a significant hydrogen bond interaction involving budesonide in the ternary mixture in mannitol, in contrast to the case of the lactose ternary mixture, for which there is little evidence of interactions.

The IR spectrum for the physical mixture formulation prepared with lactose (BTF_Lac_PM) was dominated by the bands for formoterol, despite the fact that this is the least component by weight in the formulation. Without wishing to be bound by any theory, it is believed that this result may be due to the low content uniformity of the powder formulation, which may have produced a KBr disk that was also not uniform. As a result, the portion of the KBr disk that was in the beam of the FTIR had mostly formoterol generating a spectrum similar to the spectrum of formoterol. In contrast, the physical mixture with mannitol has the carbonyl bands unchanged from the raw drugs as it was expected. A summary of the FTIR spectrum changes showing the stretching frequency shifts for the carbonyl groups of all TFF formulations is presented in Table 6.

TABLE 6
Summary of FTIR spectrum changes showing the stretching frequency shifts
for the carbonyl groups of all TFF formulations.

| | | Stretching frequency | H-bonding | | |
|---|---|---|---|---|---|
| Formulation | Chemical group | shift (cm⁻¹) | yes | no | Comments |
| Tio_Lac | Ester carbonyl | 1749 to 1729 | x | | Strong H-bonding. |
| | — | ~900 to 860 | | | Undefined band. |
| | | | | | Likely formation of H-bonding. |
| For_Lac | Fomamide carbonyl | 1687 to 1660 | x | | Market decrease in intensity. |
| | | | | | Slight frequency shift. |
| | | | | | Minimal H-bonding formation. |
| Bud_Lac | Ketone carbonyl | 1722 to 1712 | x | | Slight frequency shift. |
| | Dienone carbonyl | 1666 to 1650 | | | Minimal H-bonding formation. |
| BTF_Lac | Ketone carbonyl | 1722 to 1712 | x | | Slight frequency shift |
| | | | | | Minimal H-bonding formation. |
| Tio_Man | Ester carbonyl | 1749 to 1734 | | x | Slight frequency shift. |
| | | | | | Minimal H-bonding formation. |
| For_Man | Formamide carbonyl | 1687 to 1664 | | x | Slight frequency shift. |
| Bud_Man | Ketone carbonyl | 1723 | | x | Remains unchanged. |
| | Dienone carbonyl | 1666 | | | |
| BTF_Man | Ketone carbonyl | 1717 to 1710 | x | | Further shift to lower |
| | | | | | frequency than Bud_Man. |
| | | | | | Strong H-bonding. |
| | Dienone carbonyl | 1666 | | | Remains unchanged. |

D. In Vitro Aerosol Performance of Formulations and Deposition Homogeneity on the NGI

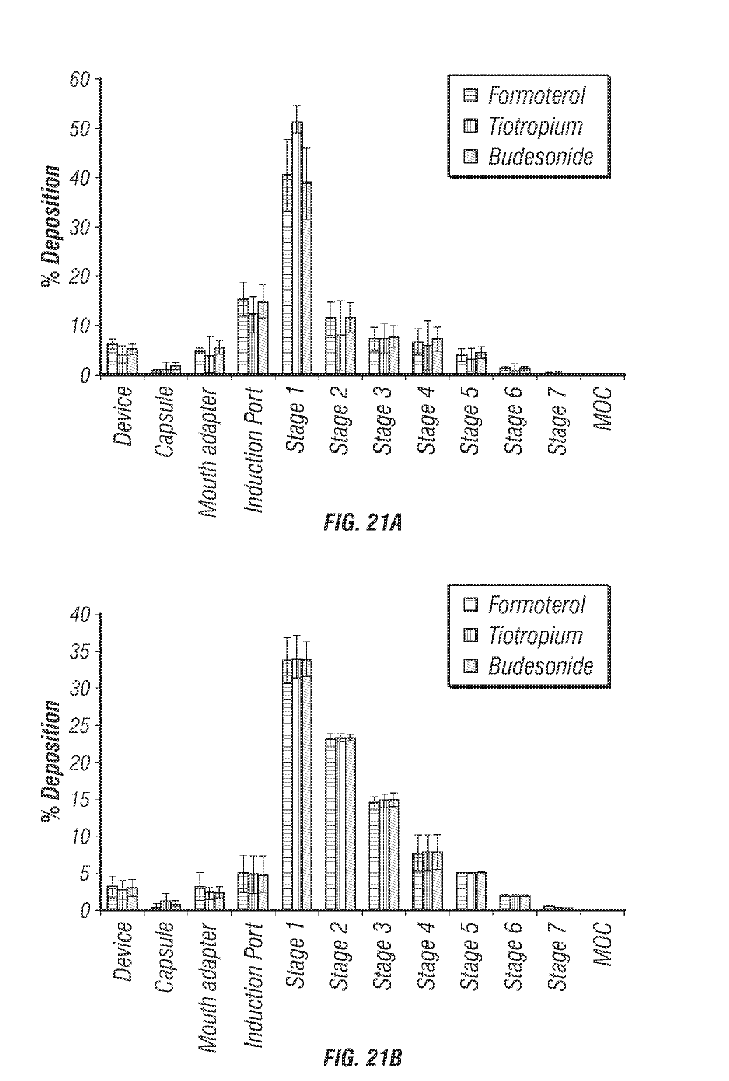

Aerodynamic particle size distribution of TFF formulations and physical mixtures were assessed using the NGI at 54 L/min and a Handihaler® dry powder inhaler device. Particle size distribution of the triple combo powder formulation prepared with lactose (BTF_Lac) is shown in FIG. 21A. Importantly, analyses of the percentage stage-by-stage distribution of all three drugs present in the formulation were not statistically different. Thin film freezing powders are prepared from a diluted drug solution, which are rapidly frozen and then freeze dried (Parikh et al., 2012). The resultant cake powder is homogenous and porous, which is easily dispersible under an inhalation air stream. Therefore, the results were in accordance with the hypothesis that each component of the formulation would be homogeneously distributed throughout the lyophilized cake and consequently homogenously dispersed at time of aerosolization. A small difference in values was noticed in the percentage FPF for all three components, which were 31.65±10.25, 26.18±0.67 and 33.08±9.19% for formoterol, tiotropium and budesonide, respectively, as seen in Table 7.

TABLE 7
Emitted dose and fine particle fraction for each drug in all triple therapy
formulations.

| Formulation | Formoterol Fumarate | Tiotropium Bromide | Budesonide |
|---|---|---|---|
| BTF_Lac | | | |
| ED | 94.68 ± 1.33 | 97.20 ± 0.70 | 92.73 ± 0.72 |
| FPF | 31.65 ± 10.25 | 26.18 ± 0.67 | 33.08 ± 9.19 |
| BTF_Man | | | |
| ED | 98.74 ± 1.69 | 98.81 ± 2.05 | 96.08 ± 1.57 |
| FPF | 52.95 ± 3.91 | 52.96 ± 3.48 | 53.60 ± 2.89 |
| BTF_Lac_PM | | | |
| ED | 69.04 ± 7.22 | 73.46 ± 6.78 | 83.68 ± 10.88 |
| FPF | 28.64 ± 3.98 | 25.22 ± 3.56 | 25.05 ± 5.82 |
| BTF_Man_PM | | | |
| ED | 85.06 ± 3.56 | 83.95 ± 3.46 | 80.00 ± 2.37 |
| FPF | 26.36 ± 3.84 | 28.65 ± 0.30 | 25.80 ± 1.96 |

Aerodynamic particle size distribution of the triple combo formulation prepared with mannitol (BTF_Man) is shown in FIG. 21B. The percentage stage-by-stage powder depositions of the three drugs present in the formulation were not statistically different. The distribution similarity is also seen in the percentage FPF values of 52.95±3.91, 52.96±3.48 and 53.60±2.89% for formoterol, tiotropium and budesonide, respectively (Table 7). BTF_Man formulation presented the greatest percentage FPF, regardless the higher specific surface area (SSA) and porosity of BTF_Lac formulation. Without wishing to be bound by any theory, it is believed that the inferior performance of BTF_Lac may be related to water sorption to the particle surfaces, which function as plasticizer on amorphous powder. In some instances, it may be desirable to manipulate the powder with lactose in a controlled low humidity environment when preparing TFF formulations. The emitted doses of the two TFF triple combo formulations were above 90% due to the fact that the brittle cake powders are easily dispersible and emitted from the dry powder inhaler and capsule (Watts et al., 2013).

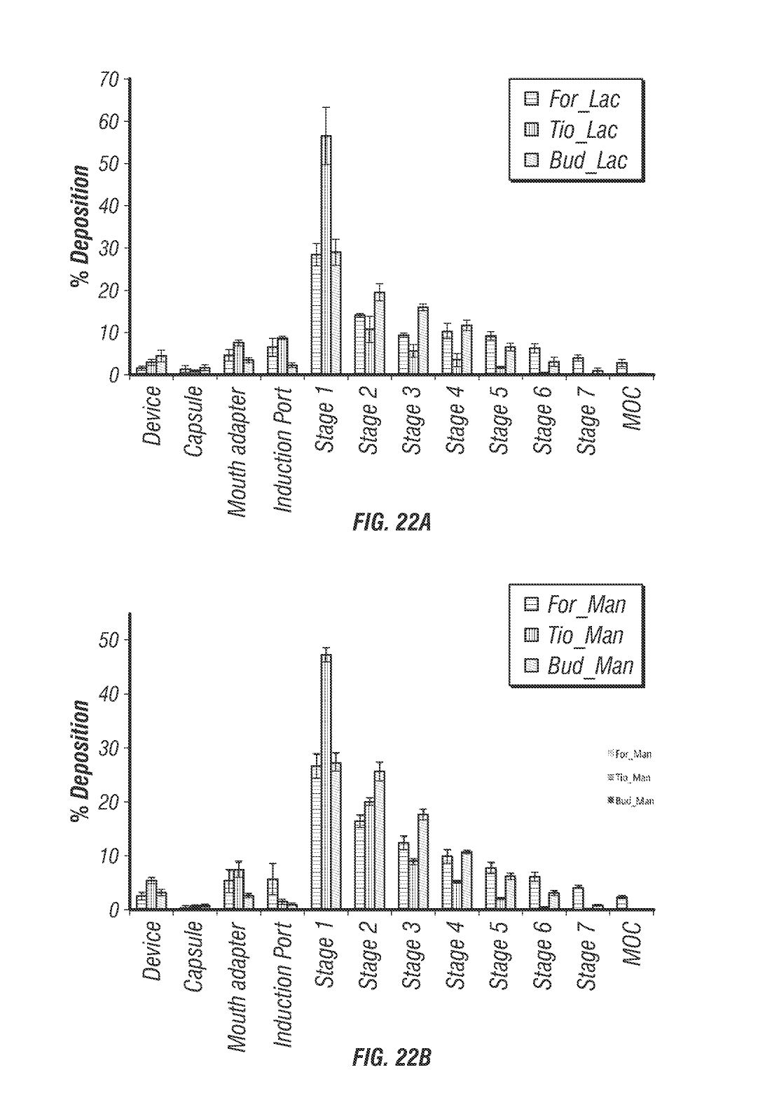

When aerosolized individually, the stage distribution and aerosol performance of each TFF formulation prepared with a single drug was statistically different. The difference is particularly significant for stage 1 where the amount of tiotropium deposited was almost double the amount of formoterol and budesonide for lactose and mannitol formulations, as seen in FIGS. 22A & 22B. The difference in aerosol performance among the single drug TFF formulations is also confirmed by the FPF values as shown in Table 8. The percentage FPF for formoterol, tiotropium and budesonide formulations (For_Lac, Tio_Lac and Bud_Lac) prepared with lactose were 55.51±5.79, 22.56±5.75 and 58.67±4.28%, respectively. Also, percentage FPF deposition of formoterol, tiotropium and budesonide prepared with mannitol (For_Man, Tio_Man and Bud_Man) were 58.32±5.99, 37.45±0.71 and 64.62±1.28%, respectively, showing a significant difference. Single drugs prepared with mannitol presented greater percentage FPF than formulations prepared with lactose. This phenomenon is in accordance with the hypothesis that hygroscopic lactose formulations are susceptible to water sorption to the powder surfaces and posterior collapse of the lyophilized cake structure. The TFF formulations prepared with tiotropium presented the smallest FPF and SSA values, which may be related to the powder physicochemical characteristics.

TABLE 8
Emitted dose and percentage fine particle
fraction of all single drug formulations.

| Formulation | ED | FPF |
|---|---|---|
| For_Lac | 96.84 ± 6.04 | 55.51 ± 5.79 |
| For_Man | 97.08 ± 0.28 | 58.32 ± 5.99 |
| Tio_Lac | 95.81 ± 0.84 | 22.56 ± 5.75 |
| Tio_Man | 93.93 ± 0.44 | 37.45 ± 0.71 |
| Bud_Lac | 93.59 ± 2.08 | 58.67 ± 4.28 |
| Bud_Man | 95.2 ± 0.68 | 64.06 ± 1.28 |

Thus, tiotropium may be responsible for the low aerosolization performance and FPF values generated by the triple combo formulations. These results imply that when patients are treated with multiple administrations of single drug formulations they may not benefit from co-deposition of drugs in the lungs and from a potential synergistic action (Sin and Man, 2007; Nelson et al., 2003). Similarly to the triple combo TFF formulations, high percentage values of emitted doses from the inhalers were seen for all single drug formulations, which is a characteristic of the TFF powders (Table 8).

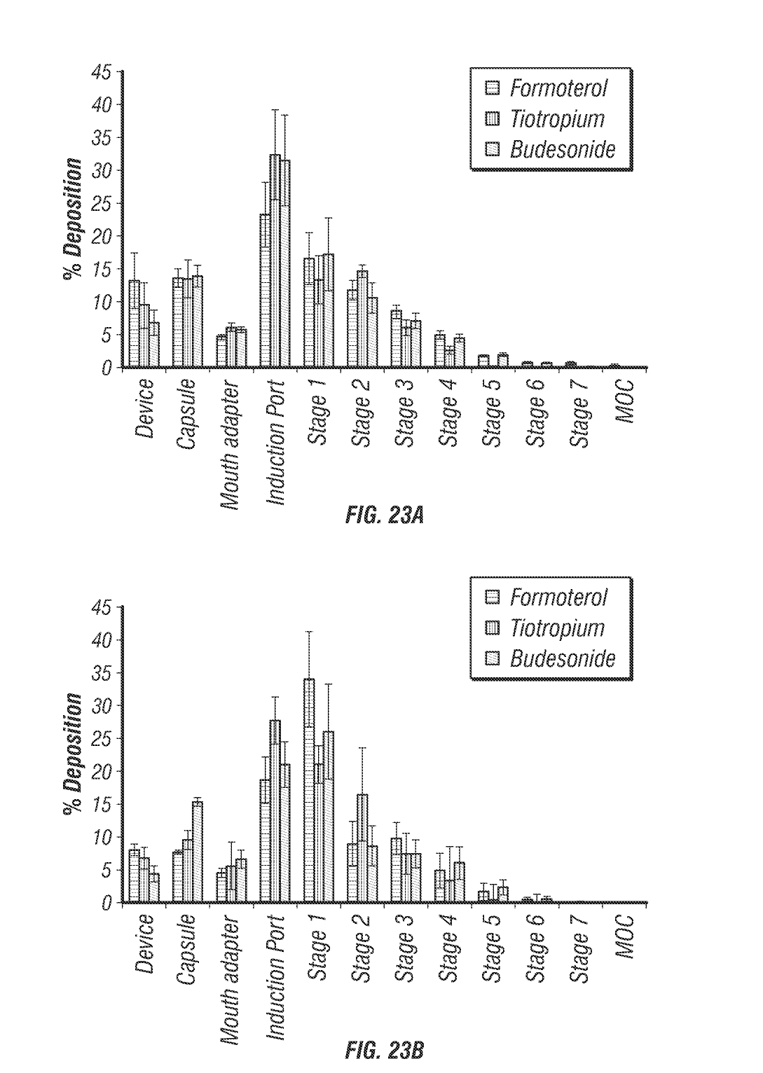

The difference in stage distribution and aerosol performance of the triple combo formulations prepared by physically blending the jet milled drug powders with coarse lactose or mannitol (BTF_Lac_PM and BTF_Man_PM) particles also were investigated. The stage-by-stage powder depositions of the BTF_Man_PM formulation were significantly different as shown in FIG. 23B. The three drugs presented different percentage deposition from each other with a high percentage of powder being deposited in the induction port. Although the percentage deposition of all three drugs on the cascade impactor stages were significantly different, the percentage FPF for all three drugs of the BTF_Man_PM formulation were similar presenting values of 26.36±03.84, 28.65±0.30 and 25.80±1.96% for formoterol, tiotropium and budesonide, respectively (Table 7). Additionally, the percentage emitted dose values reduced from above 90% to approximately 70 to 80% for BTF_Man_PM and BTF_Lac_PM due to the high amount of powder remaining in the capsules after aerosolization of both formulations, as shown in Table 7. The lower emitted dose and higher neck deposition of the physical mixture formulations may contribute to variable dosing with potential for under or overdosing. This fact is increased if the powder mixture is comprised of potent drugs (Weers et al., 2010; French et al., 1996).

The stage-by-stage powder depositions of the BTF_Lac_PM formulation were not statistically different as shown in FIG. 23A. The BTF_Lac_PM also showed low aerosolization performance presenting percentage FPF values of 28.64±3.89, 25.22±3.56 and 25.05±5.82% for formoterol, tiotropium and budesonide, respectively. However, deposition through stages 3 to 5, where particle sizes between 1-3 μm deposit and have the highest probability to reach the deep lungs, is more homogeneous for the TFF formulations than for the BTF_Lac_PM. Also, the difference of powder aerosolization performance between the physical mixtures prepared with lactose and mannitol suggests the lack of robustness of the preparation process. Without wishing to be bound by any theory, it is believed that the low aerosol performance of the physical mixture formulations may be related to the size and surface properties of the particles and result in incomplete powder dispersion and variations in aerosol performance which may influence pulmonary drug delivery (Hickey et al., 2007; Zeng et al., 1998).

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,725,871
U.S. Pat. No. 5,756,468
U.S. Pat. No. 5,780,045
U.S. Pat. No. 6,440,101
U.S. Pat. No. 8,579,855
U.S. Patent Application No. 2010/0221343
PCT Patent Application Publication No. WO 2009/125986
Alhalaweh et al., Theophylline Cocrystals Prepared by Spray Drying: Physicochemical Properties and Aerosolization Performance. AAPS PharmSciTech. 2013 Jan. 8; 14(1):265-76.
Ali, M. E. and Lamprecht, A., Spray freeze drying for dry powder inhalation of nanoparticles, European journal of pharmaceutics and biopharmaceutics: official journal of Arbeitsgemeinschaft fur Pharmazeutische Verfahrenstechnik e.V, 87 (2014) 510-517, 2014.
Berger, R. and Berger, W. E., Particle size and small airway effects of mometasone furoate delivered by dry powder inhaler, in: Allergy and Asthma Proceedings, OceanSide Publications, Inc, pp. 52-58, 2013.
Carvalho et al., Characterization and pharmacokinetic analysis of crystalline versus amorphous rapamycin dry powder via pulmonary administration in rats, European journal of pharmaceutics and biopharmaceutics: official journal of Arbeitsgemeinschaft fur Pharmazeutische Verfahrenstechnik e.V, 88:136-147, 2014.
Chougule et al., Nano-liposomal dry powder inhaler of tacrolimus: preparation, characterization, and pulmonary pharmacokinetics, International journal of nanomedicine, 2:675-688, 2007.
Chowdhury, B. A. and Pan, G. Dal, The FDA and safe use of long-acting beta-agonists in the treatment of asthma, The New England journal of medicine, 362:1169-1171, 2010.
Craig et al., An evaluation of the use of modulated temperature DSC as a means of assessing the relaxation behaviour of amorphous lactose, Pharmaceutical research, 17:696-700, 2000.
Dengale et al., Preparation and characterization of co-amorphous Ritonavir-Indomethacin systems by solvent evaporation technique: improved dissolution behavior and physical stability without evidence of intermolecular interactions, European journal of pharmaceutical sciences: official journal of the European Federation for Pharmaceutical Sciences, 62:57-64, 2014.
DiNunzio et al., Amorphous compositions using concentration enhancing polymers for improved bioavailability of itraconazole, Molecular Pharmaceutics, 5: 968-980, 2008.
Évora et al., Pyrazinamide-diflunisal: a new dual-drug co-crystal, Crystal Growth & Design, 11:4780-4788, 2011.
French et al., The influence of formulation on emission, deaggregation and deposition of dry powders for inhalation. J Aerosol Sci. 1996 July; 27(5):769-83.
Greening et al., Added salmeterol versus higher-dose corticosteroid in asthma patients with symptoms on existing inhaled corticosteroid. Allen & Hanburys Limited UK Study Group, Lancet, 344:219-224, 1994.
Heinz et al., Understanding the solid-state forms of fenofibrate—a spectroscopic and computational study, European journal of pharmaceutics and biopharmaceutics: official journal of Arbeitsgemeinschaft fur Pharmazeutische Verfahrenstechnik e.V, 71:100-108, 2009.
Hickey et al., Physical characterization of component particles included in dry powder inhalers. I. Strategy review and static characteristics. J Pharm Sci. 96(5):1282-301, 2007.
Janssens, S. and Van den Mooter, G, Review: physical chemistry of solid dispersions, The Journal of Pharmacy and Pharmacology, 61:1571-1586, 2009.
Jeffrey, G. A. An introduction to hydrogen bonding, Oxford university press New York, 1997.
Jouyban-Gharamaleki, et al., Solubility prediction of salmeterol xinafoate in water-dioxane mixtures, International Journal of Pharmaceutics, 216:33-41, 2001.
Liao, X. and Wiedmann, T. S., Solubilization of cationic drugs in lung surfactant, Pharmaceutical Research, 20:1858-1863, 2003.
Lobmann et al., A theoretical and spectroscopic study of co-amorphous naproxen and indomethacin, International journal of pharmaceutics, 453:80-87, 2013.
Lobmann et al., Co-amorphous simvastatin and glipizide combinations show improved physical stability without evidence of intermolecular interactions, European journal of pharmaceutics and biopharmaceutics: official journal of Arbeitsgemeinschaft fur Pharmazeutische Verfahrenstechnik e.V, 81:159-169, 2012.
M. JP, Practices of coating collection surfaces of cascade impactors: a survey of members of the european pharmaceutical aerosol group (EPAG), Drug Deliv Lung, 14 (2003) 4.
Mapel et al., The role of combination inhaled corticosteroid/long-acting beta-agonist therapy in COPD management, Primary care respiratory journal: journal of the General Practice Airways Group, 19:93-103, 2010.
Marple et al., Next generation pharmaceutical impactor (a new impactor for pharmaceutical inhaler testing). Part II: Archival calibration, Journal of aerosol medicine: the official journal of the International Society for Aerosols in Medicine, 16:301-324, 2003.
Morello et al., Dry-powder pulmonary insufflation in the mouse for application to vaccine or drug studies, Tuberculosis, 89:371-377, 2009.
Murnane et al., Dry powder formulations for inhalation of fluticasone propionate and salmeterol xinafoate microcrystals, Journal of Pharmaceutical Sciences, 98:503-515, 2009.
Nelson et al., Enhanced synergy between fluticasone propionate and salmeterol inhaled from a single inhaler versus separate inhalers, The Journal of Allergy and Clinical Immunology, 112:29-36, 2003.
Overhoff et al., Novel ultra-rapid freezing particle engineering process for enhancement of dissolution rates of poorly water-soluble drugs. Eur J Pharm Biopharm., 2007 January, 65(1):57-67.
Parikh et al., Improved localized lung delivery using smart combination respiratory medicines. Eur Respiratory Disease 8(1):40-45, 2012.
Patil, J. S. and Sarasija, S., Pulmonary drug delivery strategies: A concise, systematic review, Lung India: official organ of Indian Chest Society, 29:44-49, 2012.
Qian, et al., Drug-polymer solubility and miscibility: Stability consideration and practical challenges in amorphous solid dispersion development, Journal of Pharmaceutical Sciences, 99:2941-2947, 2010.
Rasenack N and Muller B W, Micron-size drug particles: Common and novel micronization techniques. Pharm Dev Technol. 9(1):1-13, 2004.
Shayanfar et al., Coamorphous atorvastatin calcium to improve its physicochemical and pharmacokinetic properties, Journal of pharmacy & pharmaceutical sciences: a publication of the Canadian Society for Pharmaceutical Sciences, Societe canadienne des sciences pharmaceutiques, 16:577-587, 2013.
Silvasti et al., a novel multidose powder inhaler—Comparison with metered dose inhaler, Drug Today, 32:11, 1996.
Simperler et al., Glass transition temperature of glucose, sucrose, and trehalose: an experimental and in silico study, The Journal of Physical Chemistry. B, 110:19678-19684, 2006.
Sin and Man, Do chronic inhaled steroids alone or in combination with a bronchodilator prolong life in chronic obstructive pulmonary disease patients? Curr Opin Pulm Med. 2007 March; 13(2):90-7.
Sowa et al., A 1:1 pharmaceutical cocrystal of myricetin in combination with uncommon piracetam conformer: X-ray single crystal analysis and mechanochemical synthesis, Journal of Molecular Structure, 1058:114-121, 2014.
Sun et al., Stability of amorphous pharmaceutical solids: crystal growth mechanisms and effect of polymer additives, The AAPS Journal, 14:380-388, 2012.
Tajber et al., Spray drying of budesonide, formoterol fumarate and their composites—I. Physicochemical characterisation. Int J Pharm. 2009 Feb. 9; 367(1-2):79-85.
Takenaga et al., J. Control Release, 52(1-2):81-87, 1998.
Trivedi et al., A Rapid, Stability-Indicating RP-HPLC Method for the Simultaneous Determination of Formoterol Fumarate, Tiotropium Bromide, and Ciclesonide in a Pulmonary Drug Product. Sci Pharm. 2012 September; 80(3):591-603.
Vehring et al., Cosuspensions of Microcrystals and Engineered Microparticles for Uniform and Efficient Delivery of Respiratory Therapeutics from Pressurized Metered Dose Inhalers. Langmuir. 2012 Oct. 23; 28(42):15015-23.
Velaga et al., Supercritical fluids crystallization of budesonide and flunisolide. Pharm Res. 2002 October; 19(10):1564-71.
Wang et al., In vitro and in vivo performance of dry powder inhalation formulations: comparison of particles prepared by thin film freezing and micronization, AAPS PharmSciTech, 15: 981-993, 2014.
Watts et al., “Respirable Low-Density Microparticles Formed In Situ from Aerosolized Brittle Matrices, Pharmaceutical Research, 30(3):813-825, 2013.
Weers et al., Pulmonary Formulations: What Remains to be Done? J Aerosol Med Pulm Drug Deliv. 2010 December; 23:S5-S23.
Weers J and Tarara T, The PulmoSphere™ platform for pulmonary drug delivery. Therapeutic delivery 5:277-295, 2014.
Williamson et al., Paradoxical trough effects of triple therapy with budesonide/formoterol and tiotropium bromide on pulmonary function outcomes in COPD. CHEST Journal; 138:595-604, 2010.
Yang et al., Pharmaceutical Cryogenic Technologies. In Formulating Poorly Water Soluble Drugs. Springer; 443-500, 2012.
Zeng et al., The role of fine particle lactose on the dispersion and deaggregation of salbutamol sulphate in an air stream in vitro. Int J Pharm. 1998 Dec. 30; 176(1):99-110.
Zitt et al., Mometasone furoate nasal spray: a review of safety and systemic effects, Drug safety, 30:317-326, 2007.

What is claimed is:

1. A pharmaceutical composition comprising either:
a. a triple therapy comprising a therapeutically effective amount of an active pharmaceutical ingredient from each of the following groups:
i. a long acting β-agonist (LABA);
ii. a long acting muscarinic antagonist (LAMA); and
iii. a cortiscosteroid (CS); or
b. a dual therapy comprising a therapeutically effective amount of an active pharmaceutical ingredient from both of the following groups:
i. a long acting β-agonist (LABA), and
ii. a corticosteroid (CS);
wherein the pharmaceutical composition is formulated as a brittle matrix particle having a specific surface area of greater than 5 m²/g.

2. The pharmaceutical composition of claim 1 further comprising one or more excipients.

3. The pharmaceutical composition of claim 2, wherein the excipient is a sugar, sugar derivative, or an amino acid.

4. The pharmaceutical composition of claim 3, wherein the excipient is a sugar or a sugar derivative.

5. The pharmaceutical composition of claim 4, wherein the excipient is lactose, mannitol, or trehalose.

6. The pharmaceutical composition of claim 2, wherein the excipient is an amino acid.

7. The pharmaceutical composition of claim 6, wherein the excipient is glycine.

8. The pharmaceutical composition according to any one of claims 1-7, wherein the long acting β-agonist is a salmeterol or formoterol salt.

9. The pharmaceutical composition of claim 8, wherein the long acting β-agonist is salmeterol xinafoate.

10. The pharmaceutical composition of claim 8, wherein the long acting β-agonist is formoterol fumarate.

11. The pharmaceutical composition according to any one of claims 1-10, wherein corticosteroid is mometasene furoate or budesomide.

12. The pharmaceutical composition according to any one of claims 1-11, wherein the long acting muscarinic antagonist is a tiotropium salt.

13. The pharmaceutical composition of claim 12, wherein the long acting muscarinic antagonist is tiotropium bromide.

14. The pharmaceutical composition according to any one of claims 1-13, wherein the dual therapy comprises a weight ratio of the long acting β-agonist to the corticosteroid from about 1:0.1 to about 1:100 in the composition.

15. The pharmaceutical composition of claim 14, wherein the dual therapy has a weight ratio of about 5:22 of the long acting β-agonist to the corticosteroid.

16. The pharmaceutical composition according to any one of claims 1-12, wherein the triple therapy comprises a weight ratio of the long acting β-agonist to the long acting muscarinic antagonist to the corticosteroid from about 1:0.1:0.1 to about 1:100:100 in the composition.

17. The pharmaceutical composition of claim 16, wherein the triple therapy weight ratio is about 1:2:35.5 of the long acting β-agonist to the long acting muscarinic antagonist to the corticosteroid.

18. The pharmaceutical composition according to any one of claims 1-12, 16, or 17, wherein the triple therapy comprises a weight ratio of the long acting β-agonist to the long acting muscarinic antagonist from about 1:0.1 to about 1:100 in the composition.

19. The pharmaceutical composition of claim 18, wherein the triple therapy weight ratio is about 1:2 of the long acting β-agonist to the long acting muscarinic antagonist.

20. The pharmaceutical composition according to any one of claims 1-12 or 16-19, wherein the triple therapy comprises a weight ratio of the long acting β-agonist to the corticosteroid from about 1:0.1 to about 1:100 in the composition.

21. The pharmaceutical composition of claim 20, wherein the triple therapy weight ratio is about 2:70 of the long acting β-agonist to the corticosteroid.

22. The pharmaceutical composition according to any one of claims 1-12 or 16-21, wherein the triple therapy comprises a weight ratio of the long acting muscarinic antagonist to the corticosteroid from about 1:0.1 to about 1:1000 in the composition.

23. The pharmaceutical composition of claim 22, wherein the triple therapy weight ratio is about 4:70 of the long acting muscarinic antagonist to the corticosteroid.

24. The pharmaceutical composition according to any one of claims 1-23, wherein the pharmaceutical composition has a molar ratio of the dual therapy or the triple therapy to the excipient of from about 1:0 to about 1:9 in the composition.

25. The pharmaceutical composition of claim 24, wherein the molar ratio is about 1:1 of the dual therapy or the triple therapy to the excipient.

26. The pharmaceutical composition according to any one of claims 1-25, wherein the pharmaceutical composition is formulated as a unit dose.

27. The pharmaceutical composition according to any one of claims 1-25, wherein the unit dose of the pharmaceutical composition comprises a dose of the long acting β-agonist from about 1 to about 500 μg.

28. The pharmaceutical composition of claim 27, wherein the dose of the long acting β-agonist is about 50 μg when the long acting β-agonist is salmeterol xinafoate.

29. The pharmaceutical composition of claim 27, wherein the dose of the long acting β-agonist is about 4.5 μg when the long acting β-agonist is formoterol fumarate.

30. The pharmaceutical composition according to any one of claims 1-25, wherein the unit dose of the pharmaceutical composition comprises a dose of the long acting muscarinic antagonist from about 1 to about 100 μg.

31. The pharmaceutical composition of claim 27, wherein the dose of the long acting muscarinic antagonist is about 9 μg.

32. The pharmaceutical composition according to any one of claims 1-25, wherein the unit dose of the pharmaceutical composition comprises a dose of the corticosteroid from about 1 to about 1000 μg.

33. The pharmaceutical composition of claim 32, wherein the dose of the corticosteroid is about 220 μg when the corticosteroid is mometasene furoate.

34. The pharmaceutical composition of claim 32, wherein the dose of the corticosteroid is about 160 μg when the corticosteroid is budesomide.

35. The pharmaceutical composition according to any one of claims 1-33, wherein the pharmaceutical composition is formulated for administration: intranasally, via aerosol, to the lungs, or via inhalation.

36. The pharmaceutical composition according to any one of claims 1-33, wherein the pharmaceutical composition is formulated for use in an inhaler.

37. The pharmaceutical composition of claim 36, wherein the inhaler is a metered dose inhaler, a dry powder inhaler, a single dose inhaler, multi-unit dose inhaler, nebulizer, or pressurized metered dose inhaler.

38. The pharmaceutical composition according to any one of claims 1-37, wherein the pharmaceutical composition is the dual therapy comprising salmeterol xinafoate and mometasene furoate.

39. The pharmaceutical composition according to any one of claims 1-37, wherein the pharmaceutical composition is the triple therapy comprising formoterol fumarate, tiotropium bromide, and budesomide.

40. The pharmaceutical composition according to any one of claims 1-39, wherein the pharmaceutical composition has a specific surface area from about 5 m²/g to about 1000 m²/g.

41. The pharmaceutical composition of claim 40, wherein the pharmaceutical composition has a specific surface area from about 10 m²/g to about 500 m²/g.

42. The pharmaceutical composition of claim 41, wherein the pharmaceutical composition has a specific surface area from about 20 m²/g to about 250 m²/g.

43. The pharmaceutical composition according to any one of claims 1-42, wherein the pharmaceutical composition has a total emitted dose (TED) of greater than 85%.

44. The pharmaceutical composition of claim 43, wherein the total emitted dose (TED) is from about 90% to about 100%.

45. The pharmaceutical composition according to any one of claims 1-44, wherein the pharmaceutical composition is free of any impurities.

46. The pharmaceutical composition according to any one of claims 1-45, wherein the pharmaceutical composition is essentially free of polyvinylpyrrolidone, polyvinylalcohol, polyacrylate, or polystyrene.

47. The pharmaceutical composition according to any one of claims 1-46, wherein the pharmaceutical composition is essentially free of any polymeric excipients.

48. The pharmaceutical composition according to any one of claims 1-47, wherein the pharmaceutical composition is essentially free of poloxamers, polyethylene glycol, or polypropylene glycol.

49. The pharmaceutical composition according to any one of claims 1-48, wherein the pharmaceutical composition is essentially free of any surfactants.

50. The pharmaceutical composition according to any one of claims 1-44, wherein the pharmaceutical composition is free of other compounds beyond the excipient and the active pharmaceutical composition.

51. A method of treating or preventing a respiratory disease or disorder in a patient in need thereof comprising administering to the patient a therapeutically effective amount of a pharmaceutical composition according to any one of claims 1-39.

52. The method of claim 51, wherein the respiratory disease or disorder is a disorder involving inflammation of the lungs or sinuses.

53. The method of claim 51, wherein the respiratory disease or disorder is asthma.

54. The method of claim 51, wherein the respiratory disease or disorder is chronic obstructive pulmonary disease.

55. The method according to any one of claims 51-54, wherein the pharmaceutical composition is administered via inhalation.

56. The method of claim 55, wherein the therapeutically effective amount is administered to the patient in one inhalation.

57. The method of claim 55, wherein the therapeutically effective amount is administered to the patient in 2 or more inhalations.

58. The method of claim 57, wherein the therapeutically effective amount is administered in 2, 3, or 4 inhalations.

59. The method according to any one of claims 51-58, wherein the method comprises administering the therapeutically effective amount to the patient once a day.

60. The method according to any one of claims 51-58, wherein the method comprises administering the therapeutically effective amount to the patient two or more times a day.

61. A method of preparing a brittle matrix pharmaceutical composition comprising:
(A) admixing two or more active pharmaceutical agents into a solvent wherein the solvent comprises an organic solvent and water to form a pharmaceutical composition wherein the pharmaceutical composition comprises an amount of the active pharmaceutical agents in the solvent from about 0.01% to about 10% (w/v);
(B) applying the pharmaceutical composition to a rotating surface wherein the surface is at a temperature from about −70° C. to about −120° C.; and
(C) freezing the pharmaceutical composition to form a brittle matrix pharmaceutical composition.

62. The method of claim 61, wherein the active pharmaceutical ingredients are selected from a long acting β-agonist, a long acting muscarinic antagonist, and a corticosteroid.

63. The method of claim 61 or claim 62, wherein the pharmaceutical composition further comprises one or more excipients.

64. The method according to any one of claims 61-63, wherein the method further comprises lyophilizing the brittle matrix pharmaceutical composition.

65. The method according to any one of claims 61-64, wherein the amount is from about 0.01% (w/v) to about 6% (w/v).

66. The method of claim 65, wherein the amount is from about 0.1% (w/v) to about 5% (w/v).

67. A pharmaceutical composition prepared according to the method of claim 61.
